(19) United States
(12) Patent Application Publication
(10) Pub. No.: US 2022/0117533 A1
(43) Pub. Date: Apr. 21, 2022

(54) METHODS AND APPARATUS FOR ACTIVATION OF A WEARABLE PATCH

(71) Applicant: Otsuka Pharmaceutical Co., Ltd., Tokyo (JP)

(72) Inventors: Tomi MATTILA, Espoo (FI); Colm MC CAFFREY, Helsinki (FI); Tapio PERNU, Espoo (FI); Mohammadhossein BEHFAR, Espoo (FI); Samuli YRJÄNÄ, Oulu (FI); Kimmo JOKELAINEN, Ii (FI); Antti TAURIAINEN, Oulu (FI); Markku VALKAMA, Oulu (FI)

(73) Assignee: Otsuka Pharmaceutical Co., Ltd., Tokyo (JP)

(21) Appl. No.: 17/436,549

(22) PCT Filed: Mar. 6, 2020
(86) PCT No.: PCT/JP2020/009815

§ 371 Date: Sep. 3, 2021

Related U.S. Application Data
(60) Provisional application No. 62/815,134, filed on Mar. 7, 2019.

(51) Int. Cl. A61B 5/257; A61B 5/00

(52) U.S. Cl. CPC A61B 5/257; A61B 5/6833

(57) ABSTRACT

In some embodiments, a system includes a patch assembly, a frame, and a conductive component. The patch assembly is configured to be coupled to a patient via an adhesive portion. The patch assembly includes an electronics subassembly. The frame has a first frame configuration in which the frame is coupled to the patch assembly via a plurality of connectors and a second frame configuration in which the plurality of connectors are broken and the frame is separated from the patch assembly. The conductive component forms a continuous loop when the frame is in the first frame configuration. A portion of the conductive component is broken when the frame is in the second frame configuration such that the conductive component is discontinuous between the first end and the second end. The portion of the conductive component is at least partially disposed on a connector from the plurality of connectors when the frame is in the first frame configuration.

### CROSS-REFERENCE TO RELATED APPLICATIONS

This application claims priority to and the benefit of U.S. Provisional Application No. 62/815,134, filed Mar. 7, 2019, entitled “Methods and Apparatus for Activation of a Wearable Patch,” the entire content of which is hereby expressly incorporated by reference for all purposes.

### TECHNICAL FIELD

Some embodiments described herein relate generally to systems, methods, and apparatus for activating a wearable patch assembly.

### BACKGROUND

Patch assemblies can be attached to a surface of a user for various purposes. For example, patch assemblies including a sensor device can be used to non-invasively measure electrical potential differences (e.g., biosignals) between locations on the skin of a human or animal to diagnose and/or monitor a condition of the human or animal. Sensor devices can also be disposed to the skin of a human or animal and be configured to communicate with implanted or digested devices (e.g., digital medicines).

Activation of patch assemblies for use on the skin of a human or animal, however, can present challenges. For example, for patch assemblies that are fully encapsulated (e.g., waterproof), traditional insulating battery tab activation mechanisms can be difficult. Additionally, for patch assemblies including a push button activation mechanism, the user may forget to activate the device. Moreover, a patch assembly may need to last an extended period of time after manufacture and prior to use. For example, the patch assembly may need to have a two to four year shelf life.

Thus, there is a need for systems, methods, and apparatus for activating wearable patch assemblies.

### SUMMARY

In some embodiments, a system includes a patch assembly, a frame, and a conductive component. The patch assembly is configured to be coupled to a patient via an adhesive portion. The patch assembly includes an electronics subassembly. The frame has a first frame configuration in which the frame is coupled to the patch assembly via a set of connectors and a second frame configuration in which the set of connectors are broken and the frame is separated from the patch assembly. The conductive component has a first end and a second end coupled to the electronics subassembly. The conductive component forms a continuous loop when the frame is in the first frame configuration. A portion of the conductive component is broken when the frame is in the second frame configuration such that the conductive component is discontinuous between the first end and the second end. The portion of the conductive component that is configured to break is at least partially disposed on a connector from the set of connectors when the frame is in the first frame configuration.

### BRIEF DESCRIPTION OF DRAWINGS

FIG. 1A is a schematic illustration of a system with a frame of the system in a first frame configuration and a second frame configuration, respectively, according to an embodiment.

FIG. 1B is a schematic illustration of a system with a frame of the system in a first frame configuration and a second frame configuration, respectively, according to an embodiment.

FIG. 1C is a schematic illustration of a system with a frame of the system in a first frame configuration and a second frame configuration, respectively, according to an embodiment.

FIG. 1D is a schematic illustration of a system with a frame of the system in a first frame configuration and a second frame configuration, respectively, according to an embodiment.

FIG. 2 is a schematic illustration of a system, according to an embodiment.

FIG. 3 is an illustration of a top view of a patch assembly, according to an embodiment.

FIG. 4 is a perspective exploded view of a patch assembly, according to an embodiment.

FIG. 5 is a perspective view of a portion of a system, according to an embodiment.

FIG. 6 is a perspective exploded view of a system, according to an embodiment.

FIG. 7 is a schematic illustration of a portion of a system, according to an embodiment.

FIG. 8 is a perspective exploded view of a system, according to an embodiment.

FIG. 9 is a perspective exploded view of a system, according to an embodiment.

FIG. 10 is a schematic illustration of the electrical components of a system, according to an embodiment.

FIG. 11 is a flow chart illustrating a method of using a system, according to an embodiment.

FIG. 12 is a schematic illustration of a system, according to an embodiment.

FIG. 13 is a perspective exploded view of a system, according to an embodiment.

### DESCRIPTION OF EMBODIMENTS

In some embodiments, a system includes a patch assembly, a frame, and a conductive component. The patch assembly is configured to be coupled to a patient via an adhesive portion. The patch assembly includes an electronics subassembly. The frame has a first frame configuration in which the frame is coupled to the patch assembly via a set of connectors and a second frame configuration in which the set of connectors are broken and the frame is separated from the patch assembly. The conductive component has a first end and a second end coupled to the electronics subassembly. The conductive component forms a continuous loop when the frame is in the first frame configuration. A portion of the conductive component is broken when the frame is in the second frame configuration such that the conductive component is discontinuous between the first end and the second end. The portion of the conductive component that is configured to break is at least partially disposed on a connector from the set of connectors when the frame is in the first frame configuration.

In some embodiments, a system includes a patch assembly and a frame assembly. The patch assembly is configured to be coupled to a patient via an adhesive portion. The patch assembly includes an electronics subassembly including a first coupling area and a second coupling area. The frame assembly has a first frame configuration in which the frame is coupled to the patch assembly via a set of connectors and a second frame configuration in which the set of connectors are broken and the frame is separated from the patch assembly. The frame assembly includes a conductive layer having a conductive frame portion, a first coupling area, and a second coupling area. The first coupling area and the second coupling area coupled to the conductive frame portion via a set of conductive connectors. Each conductive connector from the set of conductive connectors are included in a connector from the set of connectors. The first coupling area of the conductive layer is coupled to the first coupling area of the electronics subassembly. The second coupling area of the conductive layer is coupled to the second coupling area of the electronics subassembly. The conductive layer forms an electrical circuit from the first coupling area of the electronics subassembly to the second coupling area of the electronics subassembly when the frame is in the first frame configuration. Each conductive connector from the set of conductive connectors is configured to be broken when the frame is in the second frame configuration such that the conductive layer is discontinuous between the first coupling area of the conductive layer and the second coupling area of the conductive layer.

In some embodiments, a system includes a patch assembly and a protective layer. The patch assembly is configured to be coupled to a user via an adhesive portion. The patch assembly includes an electronics subassembly. The protective layer includes a conductive component. The protective layer is coupled to the adhesive portion in a first protective layer configuration and removed from the patch assembly in a second protective layer configuration. The conductive component is coupled to the electronics subassembly such that energy can be conducted from a first component of the electronics subassembly to a second component of the electronics subassembly through the conductive component in the first protective layer configuration. The conductive component is not coupled to the electronics subassembly such that less energy (e.g., no energy) is conducted from the first component of the electronics subassembly to a second component of the electronics subassembly in the second protective layer configuration.

In some embodiments, a method includes disposing a patch assembly and a frame on a surface of a user such that an adhesive portion couples the patch assembly to the surface. The patch assembly is disposed within an opening defined by a frame and coupled to the frame via a set of connectors extending between the frame and the patch. The patch assembly includes an electronic subassembly including a conductive component. A portion of the conductive component extends across a connector from the set of connectors. The connector from the set of connectors and the portion of the conductive component can be broken such that the frame is separated from the patch assembly with respect to the connector from the set of connectors. The remaining connectors from the set of connectors can be broken such that the patch assembly remains coupled to the surface and the frame is removed from the surface.

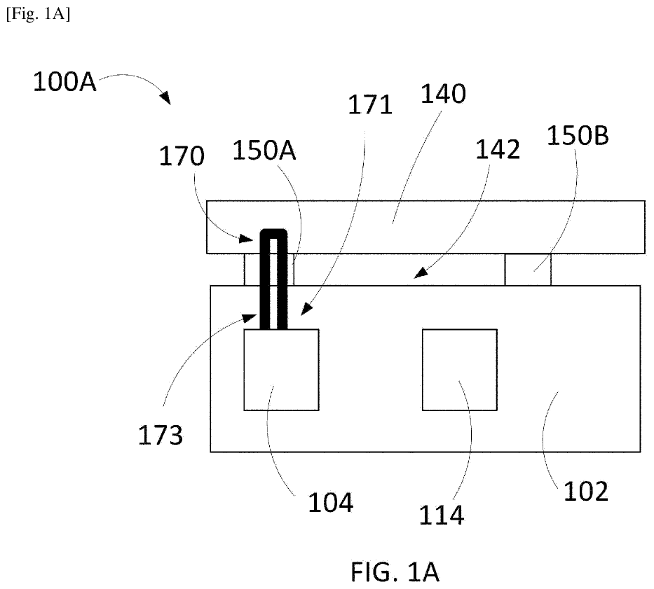

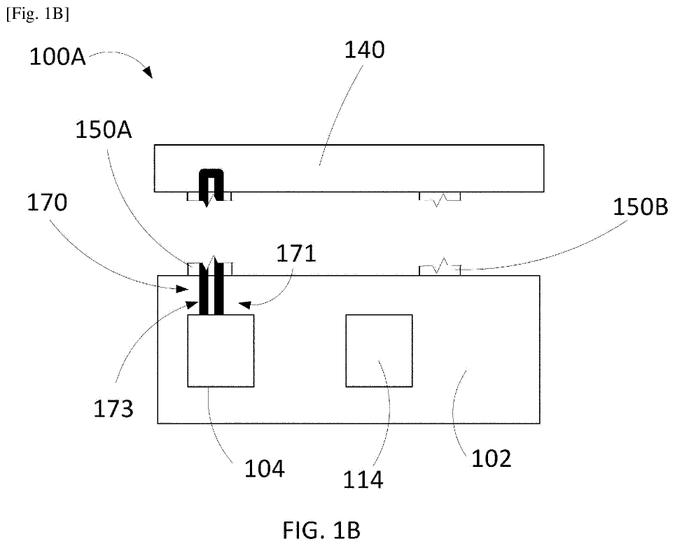

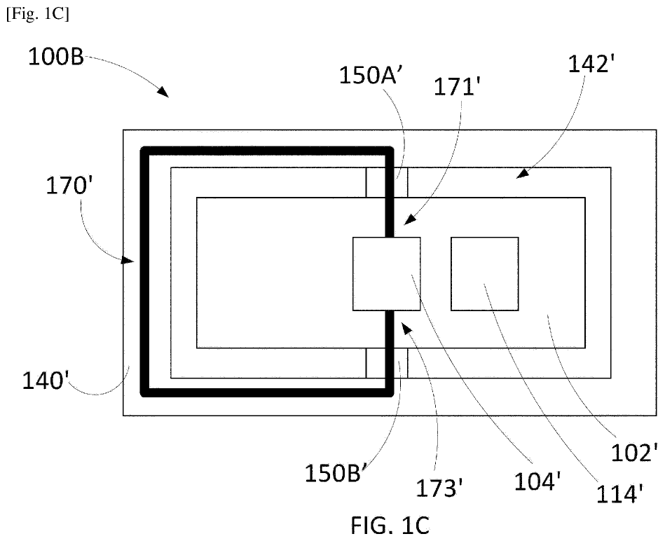

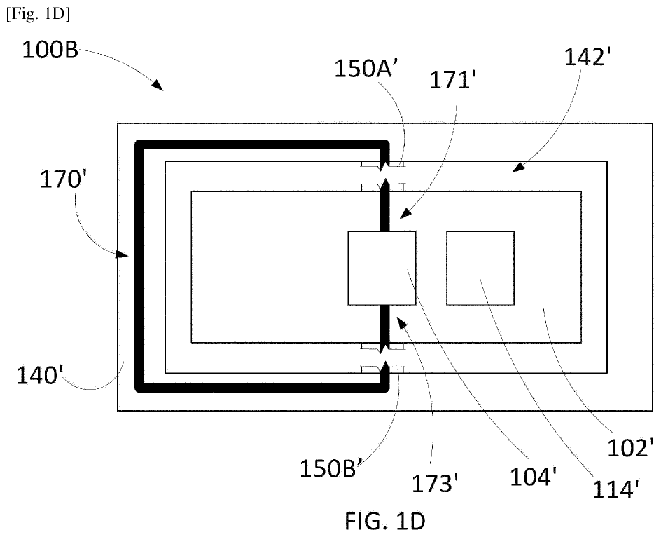

FIGS. 1A and 1B are schematic illustrations of a system 100A. The system 100A includes a patch assembly 102, a frame 140, and a conductive component 170. The patch assembly 102 is configured to be coupled to a user (e.g., a patient) via an adhesive portion 114. The patch assembly 102 includes an electronics subassembly 104. The frame 140 has a first frame configuration (shown in FIG. 1A) in which the frame 140 is coupled to the patch assembly 102 via a set of connectors 150A and 150B (collectively referred to as set of connectors 150 or connectors 150) and a second frame configuration (shown in FIG. 1B) in which the set of connectors 150 are broken and the frame 140 is separate from the patch assembly 102. The set of connectors 150 can break such that all or a portion of each connector remains attached to the frame 140 and/or the patch assembly 102 when the frame 140 is in the second frame configuration.

As shown in FIG. 1A, which shows the frame 140 in the first frame configuration relative to the patch assembly 102, the patch assembly 102 is coupled to the frame 140 via the set of connectors 150 such that a gap is defined between the patch assembly 102 and the frame 140. The set of connectors 150 includes the first connector 150A and a second connector 150B. Each connector from the set of connectors 150 extends from the frame 140 to the patch assembly 102. The conductive component 170 is coupled to and/or included in the first connector 150A and the frame 140. The portion of the conductive component 170 disposed on the first connector 150A can be configured to break when the connector is broken (e.g., during the transition of the frame 140 from the first frame configuration to the second frame configuration).

The conductive component 170 has a first end 171 and a second end 173. The first end 171 and the second end 173 are coupled to the electronics subassembly 104. The conductive component 170 forms a continuous loop and/or a closed electrical circuit when the frame 140 is in the first frame configuration. When the frame 140 is transitioned from the first frame configuration to the second frame configuration, the conductive component 170 can be configured to break at the location of or near a first connector 150A to which the conductive component 170 is coupled. When the frame 140 is in the second frame configuration, the conductive component 170 is broken such that the conductive component 170 is discontinuous between the first end 171 and the second end 173 and does not form an electrical circuit.

The electronics subassembly 104 can include a composite assembly that can be included in and/or otherwise form an integrated circuit (IC), a printed circuit board (PCB) assembly including a printed circuit board, an application-specific integrated circuit (ASIC), or any other suitable electrical circuit structure. The electronics sub-assembly 104 can include any suitable electronic components such as, for example, one or more electrodes, a processor, a memory, and/or an energy storage device (e.g., a battery, a capacitor, etc.). In some embodiments, the conductive component 170 can include a first portion of a PCB and can be coupled to the electronics subassembly 104, which includes a second portion of a PCB. In some embodiments, the conductive component 170 can include a printed conductor material.

The electronics subassembly 104 can be configured to detect when the conductive component 170 is discontinuous (e.g., fragmented due to being broken in an area proximate a connector from the set of connectors 150). For example, when the frame 140 is in the first frame configuration, a portion of the electronics subassembly 104 can detect a voltage below a threshold (e.g., based on the conductive component 170 forming a closed circuit). This can keep the electronics subassembly 104 in a sleep and/or inactive state. For another example, the electronics subassembly 104 can transmit electrical energy and/or current from the electronics subassembly 104 (e.g., from the energy storage device), through the first end 171 of the conductive component 170, through the conductive component 170, through the second end 173 of the conductive component 170 and to the electronics subassembly 104 such that the energy and/or current is transmitted through a closed circuit. The electronics subassembly 104 can be configured to detect that electrical energy and/or current is flowing through the conductive component 170 such that the conductive component 170 forms a continuous loop. For example, when the frame 140 is in the first frame configuration, a low voltage can be constantly, periodically and/or sporadically applied to the conductive component 170 to identify that the patch assembly 102 is connected to the frame 140. This can keep the remaining portions of the electronics subassembly 104 on the patch assembly 102 in a sleep and/or inactive state.

When the frame 140 is in the second frame configuration shown in FIG. 1B, the connectors from the set of connectors 150 and portions of the conductive component 170 coupled to the connectors 150 are broken. Thus, energy and/or current is unable to be transmitted by the electronics subassembly 104 through the first end 171 to the second end 173 of the conductive component 170 due to the conductive component 170 being discontinuous in the portions broken during separation of the frame 140 from the patch assembly 102 (e.g., the conductive component 170 forms an open circuit when the frame 140 is in the second frame configuration). In some instances, when the conductive component 170 is broken, the voltage at the portion of the electronics subassembly 104 can increase, activating the electronics subassembly. In other instances, the electronics subassembly 104 can be configured to detect that the conductive component 170 is discontinuous due to the energy and/or current flowing through the first end 171 not being received by the electronics subassembly 104 via the second end 173. For example, in response to the electronics subassembly 104 identifying that the conductive component 170 is broken and no longer forms a closed electrical circuit, the remaining portions of the electronics subassembly 104 can transition to an active state (e.g., to begin monitoring and/or sensing biological parameters of the user via electrodes).

In response to determining that the conductive component 170 is discontinuous, the electronics subassembly 104 can be configured to actuate a component and/or operation of the electronics subassembly 104. In some implementations, the energy and/or current provided to actuate a component and/or operation of the electronics sub-assembly 104 can be provided at a greater power level than the power level of the energy and/or current provided to the conductive component 170 prior to the conductive component 170 being broken. For example, in some embodiments in which the electronics subassembly 104 includes a number of electrodes configured to be coupled to a surface (e.g., skin) of a user, the electronics subassembly 104 can actuate a component and/or operation of the electronics subassembly 104 to measure electrical potential differences between locations on the surface to which the electrodes are coupled. In some embodiments, the electronics subassembly 104 can include a sensor that can be actuated in response to the conductive component 170 being broken. In some embodiments, the electronics subassembly 104 can be actuated to detect signals (e.g., electrocardiogram (EKG) signals, electroencephalogram (EEG) signals, electromyography (EMG) signals), to detect digital medicine within a user (e.g., wearer) of the patch assembly 102, to detect signals from an ingested, implanted, or inserted device within a user, and/or to transmit information to an external communication device (e.g., a smart phone) via any suitable communication method (e.g., Bluetooth(trademark), Near-Field Communication (NFC), or WiFi(trademark)).

In some embodiments, the patch assembly 102 can have a first patch configuration and a second patch configuration. For example, the patch assembly 102 or a portion of the patch assembly 102 may have a different shape (e.g., outer profile) and/or a different length in the first patch configuration compared to the second patch configuration. When the frame 140 is in the first frame configuration relative to the patch assembly 102, the frame 140 can maintain the patch assembly 102 in the first patch configuration via the connectors 150. In some embodiments, the patch assembly 102 or a portion of the patch assembly 102 can be elastic and/or flexible. In some embodiments, the patch assembly 102 can be biased toward the first patch configuration. The frame 140 can be substantially inelastic. For example, the frame 140 can be inelastic along its longitudinal axis such that, when in the first frame configuration when the frame 140 is coupled to the patch assembly 102, the frame 140 (via the connectors 150) can prevent the patch assembly 102 from changing shape. For example, the frame 140 can prevent the patch assembly 102 from transitioning between the first patch configuration and the second patch configuration without deliberate intervention from the user.

Although the system 100 is shown as having two connectors 150 in FIGS. 1A and 1B, in some embodiments, the system 100 can include any suitable number of connectors 150 (e.g., three, four, five, six, seven, eight, nine, ten or more connectors) arranged in any suitable arrangement. In some embodiments, the connectors 150 can have any suitable size or shape such that the connectors 150 can be broken via applying a force to the frame 140 (e.g., by pulling on the frame 140) such that the frame 140 is separated from the patch assembly 102. For example, the connectors 150 can be shaped as rectangular or triangular segments. The connectors 150 can have a first end coupled to the frame 140 and a second end coupled to the patch assembly 102 and can be tapered from the frame 140 to the patch assembly 102 or from the patch assembly 102 to the frame 140. In some embodiments, a larger portion of an outer perimeter of the patch assembly 102 is free from the connectors 150 than a total portion of the outer perimeter of the patch assembly 102 coupled to a connector 150 from the set of connectors 150. In some embodiments, rather than including a number of discrete connectors 150, an interface between the frame 140 and the patch assembly 102 can include perforations such that the frame 140 and the patch assembly 102 can be separated via breaking the perforations. In some embodiments, each connector 150 can be shaped and sized such that the force used to break each connector 150 and breakable portion of the conductive component 170 coupled to each connector 150 or to simultaneously break a number of connectors 150, some of which may be coupled to a breakable portion of the conductive component 170, is less than the force on the patch assembly 102 that would separate the patch assembly 102 from a surface of a user to which the patch assembly 102 is coupled via the adhesive portion 114.

In use, the system 100 can be coupled to a surface of a user via the adhesive portion 114 with the frame 140 in the first frame configuration relative to the patch assembly 102. In some implementations, the patch assembly 102 can be in the first patch configuration. In some implementations, a portion of the electronics subassembly 104 can detect a voltage below a threshold (e.g., based on the conductive component 170 forming a closed circuit). This can keep the electronics subassembly 104 in a sleep state. In other implementations, the electronics subassembly 104 of the patch assembly 102 can provide energy and/or current from an energy storage device of the patch assembly 102 through the conductive component 170 in a continuous loop configuration. With the patch assembly 102 coupled to the user via the adhesive portion 114, the frame 140 can be separated from the patch assembly 102 via breaking the connectors 150. For example, the frame 140 can be pulled away from the surface of the user and the patch assembly 102 with a force great enough to break the interfaces between each of the connectors 150 and the patch assembly 102. The force used to break each of the connectors 150 can be sufficiently low such that pulling the frame 140 away from the patch assembly 102 breaks the set of connectors 150 and the portions of the conductive component 170 coupled to or included in a connector from the set of connectors 150 (e.g., at the interface between each of the connectors 150 and the patch assembly 102) but does not disrupt the adhesive interface between the adhesive portion 114 and the surface of the user such that the patch assembly 102 remains coupled to the surface of the user during and after the separation of the frame 140 from the patch assembly 102. Upon the conductive component 170 being broken with the breaking of the connectors 150 such that the conductive component 170 is discontinuous, the electronics subassembly 104 can detect that the conductive component 170 is discontinuous. In response to detecting that the conductive component 170 is discontinuous, the electronics subassembly 104 can activate another component(s) and/or operation(s) of the electronics subassembly 104. In some implementations, the electronics subassembly 104 can provide energy and/or current to the other component(s) of the electronics subassembly 104 at a higher power level when in the second patch configuration than the power level of the energy and/or current provided to the conductive component 170 when in the first patch configuration. In some implementations, the patch assembly 102 can then transition from the first patch configuration to the second patch configuration while remaining coupled to the surface of the patient via the adhesive portion 114.

FIGS. 1C and 1D are schematic illustrations of a system 100B. Portions of the system 100B can be the same or similar in structure and/or function to any of the systems described herein, such as the system 100A. For example, the system 100B includes a patch assembly 102′, a frame 140′, and a conductive component 170′. The patch assembly 102′ is configured to be coupled to a user (e.g., a patient) via an adhesive portion 114′. The patch assembly 102′ includes an electronics subassembly 104′. The frame 140′ has a first frame configuration (shown in FIG. 1C) in which the frame 140′ is coupled to the patch assembly 102′ via a set of connectors 150A′ and 150B′ (collectively referred to as set of connectors 150′ or connectors 150′) and a second frame configuration (shown in FIG. 1D) in which the set of connectors 150′ are broken and the frame 140′ is separate from the patch assembly 102′. The set of connectors 150′ can break such that all or a portion of each connector remains attached to the frame 140′ and/or the patch assembly 102′ when the frame 140′ is in the second frame configuration.

As shown in FIG. 1C, which shows the frame 140′ in the first frame configuration relative to the patch assembly 102′, the patch assembly 102′ is disposed within an opening 142′ defined in the frame 140′ such that a gap is defined between the patch assembly 102′ and the frame 140′. The set of connectors 150′ includes a first connector 150A′ and a second connector 150B′. Each connector from the set of connectors 150′ extends from the frame 140′ to the patch assembly 102′. The conductive component 170′ is coupled to and/or included in the first connector 150A′, the frame 140′, and the second connector 150B′. For example, the conductive component 170′ can include a first segment included in the first connector 150A′, a second segment disposed on the frame 140′, and a third segment included in the second connector 150B′. The segment of the conductive component 170′ disposed on a connector from the set of connectors 150′ can be configured to break when that connector 150A′ or 150B′ is broken (e.g., during the transition of the frame 140′ from the first frame configuration to the second frame configuration).

The conductive component 170′ has a first end 171′ and a second end 173′. The first end 171′ and the second end 173′ are coupled to the electronics subassembly 104′. The conductive component 170′ forms a continuous loop and/or a closed electrical circuit when the frame 140′ is in the first frame configuration. When the frame 140′ is transitioned from the first frame configuration to the second frame configuration, the conductive component 170′ can be configured to break at the location of or near a first connector 150A′ and a second connector 150B′ to which the conductive component 170′ is coupled. When the frame 140′ is in the second frame configuration, at least a portion of the conductive component 170′ is broken such that the conductive component 170′ is discontinuous between the first end 171′ and the second end 173′ and no longer forms a closed electrical circuit. The portion of the conductive component 170′ can be disposed on a first connector 150A′ and/or a second connector 150B′ from the set of connectors 150′.

The electronics subassembly 104′ can include a composite assembly that can be included in and/or otherwise form an integrated circuit (IC), a printed circuit board (PCB) assembly including a printed circuit board, an application-specific integrated circuit (ASIC), or any other suitable electrical circuit structure. The electronics subassembly 104′ can include any suitable electronic components such as, for example, one or more electrodes, a processor, a memory, and/or an energy storage device (e.g., a battery).

The electronics subassembly 104′ can be configured to detect when the conductive component 170′ is discontinuous (e.g., fragmented due to being broken in an area proximate a connector from the set of connectors 150′) and no longer forms a closed electrical circuit. For example, when the frame 140′ is in the first frame configuration, a portion of the electronics subassembly 104′ can detect a voltage below a threshold (e.g., based on the conductive component 170′ forming a closed circuit). This can keep the electronics subassembly 104′ in a sleep state. For another example, the electronics subassembly 104 can transmit electrical energy and/or current from the electronics subassembly 104′ (e.g., from the energy storage device), through the first end 171′ of the conductive component 170′, through the conductive component 170′, through the second end 173′ of the conductive component 170 and to the electronics subassembly 104′ such that the energy and/or current is transmitted through a closed circuit. The electronics subassembly 104′ can be configured to detect that electrical energy and/or current is flowing through the conductive component 170′ such that the conductive component 170′ forms a continuous loop and/or closed electrical circuit.

When the frame 140′ is in the second frame configuration shown in FIG. 1D, the connectors from the set of connectors 150′ and portions of the conductive component 170′ coupled to the connectors 150′ are broken. In some instances, when the conductive component 170′ is broken, the voltage at the portion of the electronics sub-assembly 104′ can increase, activating the electronics subassembly. In other instances, the energy and/or current transmitted by the electronics subassembly 104′ through the first end 171′ of the conductive component 170′ is not able to travel to the second end 173′ of the conductive component 170′ due to the conductive component 170′ being discontinuous in the portions broken during separation of the frame 140′ from the patch assembly 102′ (e.g., the conductive component 170′ forms an open circuit when the frame 140′ is in the second frame configuration). The electronics subassembly 104′ can be configured to detect that the conductive component 170′ is discontinuous due to the energy and/or current flowing through the first end 171′ not being received by the electronics subassembly 104′ via the second end 173′.

In response to determining that the conductive component 170′ is discontinuous, the electronics subassembly 104′ can be configured to actuate a component and/or operation of the electronics subassembly. In some implementations, the energy and/or current provided to actuate a component and/or operation of the electronics sub-assembly can be provided at a higher power level than the power level of the energy and/or current provided to the conductive component 170′ prior to the conductive component 170′ being broken. For example, in some embodiments in which the electronics subassembly 104′ includes a number of electrodes configured to be coupled to a surface (e.g., skin) of a user, the electronics subassembly 104′ can actuate a component and/or operation of the electronics subassembly to measure electrical potential differences between locations on the surface to which the electrodes are coupled.

In some embodiments, the patch assembly 102′ can have a first patch configuration and a second patch configuration. For example, the patch assembly 102′ or a portion of the patch assembly 102′ may have a different shape (e.g., outer profile) and/or a different length in the first patch configuration compared to the second patch configuration. When the frame 140′ is in the first frame configuration relative to the patch assembly 102′, the frame 140′ (via the connectors 150′) can maintain the patch assembly 102′ in the first patch configuration. In some embodiments, the patch assembly 102′ or a portion of the patch assembly 102′ can be elastic and/or flexible. In some embodiments, the patch assembly 102′ can be biased toward the first patch configuration. The frame 140′ can be substantially inelastic. For example, the frame 140′ can be inelastic along its longitudinal axis such that, when in the first frame configuration when the frame 140′ is coupled to the patch assembly 102′, the frame 140′ can prevent the patch assembly 102′ from changing shape via the connectors 150′. For example, the frame 140′ can prevent the patch assembly 102′ from transitioning between the first patch configuration and the second patch configuration without deliberate intervention from the user.

In some embodiments, the shape of the opening 142′ of the frame 140′ can correspond to the shape of the patch assembly 102′ or a portion of the patch assembly 102′. For example, the patch assembly 102′ can include a connecting member (not shown in FIG. 1C) joining a first portion of the patch assembly 102′ and a second portion of the patch assembly 102′. The connecting member can have a first configuration when the patch assembly 102′ is in the first patch configuration and a second configuration when the patch assembly 102′ is in the second patch configuration. The connecting member can include a first segment coupled to a second segment via a flexible hinge. The first segment and the second segment can be arranged at a first angle when the connecting member is in the first configuration. The opening 142′ of the frame 140′ can include a first opening portion configured to receive the first segment and a second opening portion configured to receive the second segment. The first opening portion can be disposed at a second angle relative to the second opening portion and the second angle can be the same as the first angle. In some embodiments, a connecting member of the patch assembly 102′ can have a first sinusoidal shape having a first frequency in the first configuration and a second sinusoidal shape having a second frequency in the second configuration. The second frequency can be different from the first frequency. The opening 142′ of the frame 140′ can include an opening portion having a sinusoidal shape having the first frequency. In some embodiments, the connecting member can be biased toward the first configuration of the connecting member.

Although the system 100B is shown as having two connectors 150′ in FIGS. 1C and 1D, in some embodiments, the system 100B can include any suitable number of connectors 150′ (e.g., three, four, five, six, seven, eight, nine, ten or more connectors) arranged in any suitable arrangement. For example, while FIG. 1C shows two connectors 150′ each coupled to or including a portion of the conductive component 170′, the system 100B can include additional connectors coupling the frame 140′ to the patch assembly 102′ in the first frame configuration, each of the additional connectors not including or coupled to a portion of the conductive component 170′. In some embodiments, the connectors 150′ can have any suitable size or shape such that the connectors 150′ can be broken via applying a force to the frame 140′ (e.g., by pulling on the frame 140′) such that the frame 140′ is separated from the patch assembly 102′. For example, the connectors 150′ can be shaped as rectangular or triangular segments. The connectors 150′ can have a first end coupled to the frame 140′ and a second end coupled to the patch assembly 102′ and can be tapered from the frame 140′ to the patch assembly 102′ or from the patch assembly 102′ to the frame 140′. In some embodiments, a larger portion of an outer perimeter of the patch assembly 102′ is free from the connectors 150′ than a total portion of the outer perimeter of the patch assembly 102′ coupled to a connector 150′ from the set of connectors 150′. In some embodiments, rather than including a number of discrete connectors 150′, an interface between the frame 140′ and the patch assembly 102′ can include perforations such that the frame 140′ and the patch assembly 102′ can be separated via breaking the perforations. In some embodiments, each connector 150′ can be shaped and sized such that the force used to break each connector 150′ and breakable portion of the conductive component 170′ coupled to each connector 150′ or to simultaneously break a number of connectors 150′, some of which may be coupled to a breakable portion of the conductive component 170′, is less than the force on the patch assembly 102′ that would separate the patch assembly 102′ from a surface of a user to which the patch assembly 102′ is coupled via the adhesive portion 114′.

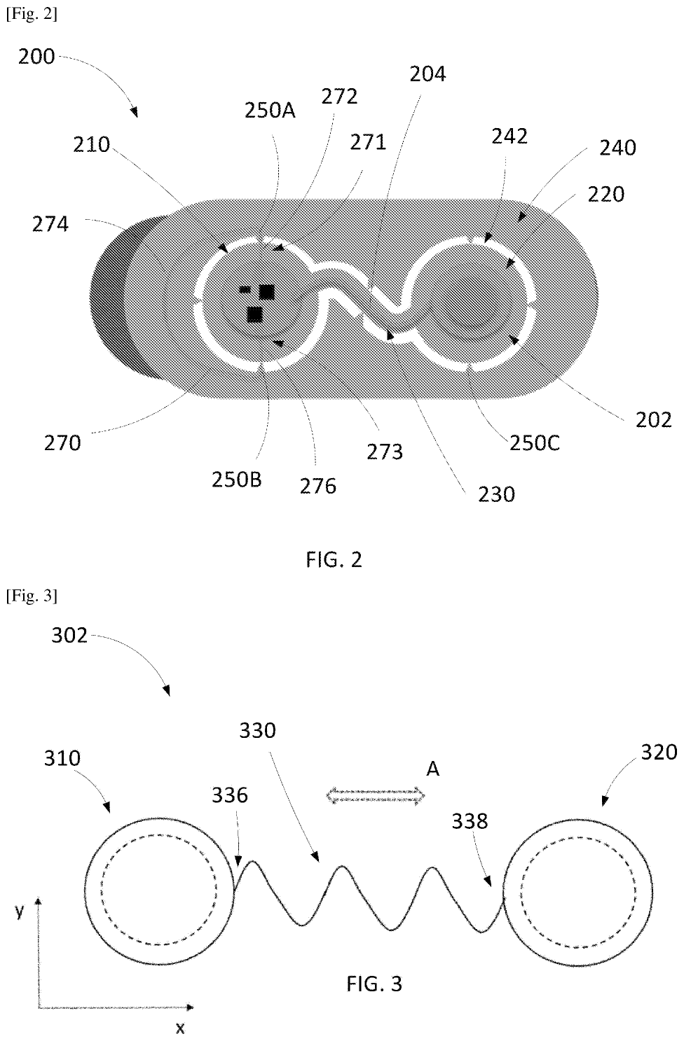

FIG. 2 is a schematic illustration of a system 200. Portions of the system 200 can be the same or similar in structure and/or function to any of the systems described herein, such as the system 100A and/or the system 100B described above. For example, the system 200 includes a patch assembly 202, a frame 240, a group of connectors including connectors 250A, 250B, 250C and also referred to as connectors 250, and a conductive component 270. The patch assembly 202 can include a first assembly 210, a second assembly 220, and a connector assembly 230. The patch assembly 202 can be disposed within an opening 242 defined by the frame 240 such that a gap is defined between the patch assembly 202 and the frame 240. The patch assembly 202, the frame 240, the group of connectors 250, and the conductive component 270 can be the same or similar in structure and/or function to the patch assembly 102 or 102′, the frame 140 or 140′, the group of connectors 150 or 150′, and the conductive component 170 or 170′, respectively, described above with reference to the systems 100A and 100B of FIGS. 1A-1D, respectively. The frame assembly 240 can have a first frame configuration in which the frame assembly 240 is coupled to the patch assembly 202 via the group of connectors 250 (e.g., connector 250A, connector 250B, connector 250C) and a second frame configuration in which the group of connectors 250 are broken and the frame assembly 240 is separated from the patch assembly 202. The patch assembly 202 can include an electronics subassembly 204 that includes a composite assembly. The composite assembly can include, for example, a flexible PCB. The conductive component 270 can be electrically coupled to the electronics subassembly 204.

The conductive component 270 has a first end 271 and a second end 273. The first end 271 and the second end 273 are coupled to the electronics subassembly 204. The conductive component 270 forms a continuous loop and/or a closed electrical circuit with the electronics subassembly 204 when the frame 240 is in the first frame configuration such that energy and/or current can be sent through the first end 271 and be received by the second end 273. In some instances, this can cause a voltage at a portion of the electronics subassembly 204 to detect a voltage below a threshold. When the frame 240 is transitioned from the first frame configuration to the second frame configuration, the conductive component 270 can be configured to break at the location of or near a first connector 250A and a second connector 250B to which the conductive component 270 is coupled. When the frame 240 is in the second frame configuration, at least a portion of the conductive component 270 is broken such that the conductive component 270 is discontinuous between the first end 271 and the second end 273 and no longer forms a closed electrical circuit. For example, as shown in FIG. 2, the conductive component 270 can include a first segment 272 included in the first connector 250A, a second segment 274 disposed on the frame 240, and a third segment 276 included in the second connector 150B. The portion of the first segment 272 disposed on the first connector 250A and the portion of the third segment 276 disposed on the second connector 250B can be configured to break when the first connector 250A or the second connector 250B, respectively, is broken (e.g., during the transition of the frame 240 from the first frame configuration to the second frame configuration).

In some implementations, the patch assembly 202 can have a first patch configuration and a second patch configuration. For example, the patch assembly 202 or a portion of the patch assembly 202 may have a different shape (e.g., outer profile) and/or a different length in the first patch configuration compared to the second patch configuration. When the frame assembly 240 is in the first frame configuration relative to the patch assembly 202, the frame assembly 240 can maintain the patch assembly 202 in the first patch configuration via the group of connectors 250. The frame assembly 240 can be substantially inelastic. For example, the frame assembly 240 can be substantially inelastic along its longitudinal axis such that, when in the first frame configuration when the frame assembly 240 is coupled to the patch assembly 202, the frame assembly 240 (via the group of connectors 250) can prevent the patch assembly 202 from changing shape. For example, the frame assembly 240 can prevent the patch assembly 202 from transitioning between the first patch configuration and the second patch configuration.

The patch assembly 202 can include a housing (not shown in FIG. 2) and an adhesive portion (not shown in FIG. 2). In some embodiments, the housing can include an upper housing portion (not shown in FIG. 2). In some embodiments, the housing can include an upper housing portion and/or a lower housing portion (not shown in FIG. 2). The frame assembly 240 includes a top layer (not shown in FIG. 2). The top layer is coupled to the patch assembly 202 in a first frame configuration via the connectors 250. For example, the top layer can be coupled to the housing via the connectors 250. In some embodiments, the top layer, the connectors 250, and the housing can be formed of the same material. In some embodiments, the top layer, the connectors 250, and the housing can be monolithically or integrally formed (e.g., formed of one sheet of material). In a second frame configuration, the frame assembly 240 can be separated from the patch assembly 202 via breaking the connectors 250.

In some embodiments, any of the frames described herein can be the same or similar to any of the frames or frame assemblies described in U.S. Provisional Patent Application No. 62/815,137, filed Mar. 7, 2019, titled “Methods and Apparatus for a Frame Surrounding a Wearable Patch,” which is incorporated by reference herein in its entirety. In some embodiments, any of the patch assemblies described herein can be the same or similar to any of the patch assemblies described in International Patent Application No. PCT/JP2020/002521, filed Jan. 24, 2020 and titled “Elastic Wearable Sensor” (hereinafter “the '521 application”) and/or U.S. Provisional Patent Application No. 62/796,435, filed Jan. 24, 2019, titled “Elastic Wearable Sensor,” each of which is incorporated by reference herein in its entirety. For example, any of the patch assemblies described herein can include patch assembly 302 shown in FIG. 3. FIG. 3 is a schematic illustration of a top view of the patch assembly 302. The patch assembly 302 can include a first assembly 310, a second assembly 320, and a connecting member 330. As shown in FIG. 3, the connecting member 330 includes a first end 336 and a second end 338. The connecting member 330 is coupled to the first assembly 310 via the first end 336 and to the second assembly 320 via the second end 338. The connecting member 330 is configured to transition between a first configuration (shown in FIG. 3) and a second configuration in which the first assembly 310 and the second assembly 320 are a different distance away from each other than in the first configuration. For example, when the patch assembly 302 is coupled to a patient's skin, a force (e.g., due to deformation due to skin flexing or tension) may be applied to the first assembly 310 and/or the second assembly 320 in either direction represented by the double-ended arrow A (e.g., in the X-direction) such that the length of the connecting member 330 from the first end 336 to the second end 338 is increased or decreased and the connecting member 330 is compressed or expanded. In some implementations, a force may be applied to the first assembly 310 and/or the second assembly 320 in a direction in the X-Y plane such that the length of the connecting member 330 from the first end 336 to the second end 338 is increased or decreased and the connecting member 330 is compressed or expanded. In some embodiments, the connecting member 330 can be biased toward the first configuration of the connecting member 330.

A frame assembly, such as any of the frame assemblies described herein, can be coupled to the patch assembly 302 via connectors coupled to the first assembly 310, the second assembly 320, and/or the connecting member 330 such that the frame assembly can maintain the patch assembly 302 in a first patch configuration. For example, the frame assembly can maintain an intended distance between the first assembly 310 and the second assembly 320 such that when the patch assembly 302 is coupled to a user's skin via adhesive, the first assembly 310 and the second assembly 320 are separated by the intended distance. For instance, the first assembly 310 and the second assembly 320 may each include an electrode, and the frame assembly may be configured to maintain an electrode to electrode distance of the patch assembly 302 such that the electrodes can be properly spaced apart when the patch assembly 302 is coupled to a user's skin. Additionally, in some embodiments, an opening defined by the frame assembly can be shaped to correspond to an outer profile of at least a portion of the patch assembly disposed within the opening. For example, the connecting member 330 may have a sinusoidal shape and the opening defined by the frame assembly can have a sinusoidal shape corresponding to an outer profile of at least a portion of the connecting member 330.

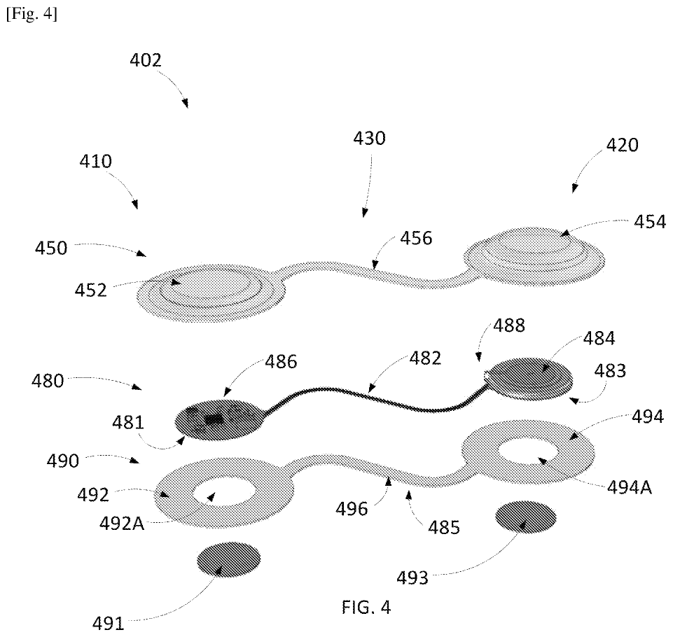

In some embodiments, any of the patch assemblies described herein can include patch assembly 402 shown in FIG. 4. FIG. 4 is a perspective exploded view of a patch assembly 402. Portions of the patch assembly 402 can be the same or similar in structure and/or function to any of the patch assemblies described herein, such as, for example, the patch assembly 102 (of FIG. 1A), the patch assembly 102′ (of FIG. 1C), the patch assembly 202 (of FIG. 2), or the patch assembly 302 (of FIG. 3). The patch assembly 402 includes a first assembly 410, a second assembly 420, and a connecting member 430 that can be the same or similar in structure and/or function, for example, to the first assembly 310, the second assembly 320, and/or the connector 330, respectively. The first assembly 410 includes a first upper housing 452, a portion 486 of a composite assembly 480, a first lower housing 492, and a first adhesive portion (not shown). The composite assembly 480 can be included in and/or otherwise form an integrated circuit (IC), a printed circuit board (PCB) assembly including a printed circuit board, an application-specific integrated circuit (ASIC), or any other suitable electrical circuit structure. For example, the portion 486 can include any suitable electronic components (e.g., a processor and a memory). The first lower housing 492 defines an opening 492A such that an electrode 481 disposed on a bottom side of the portion 486 is accessible through the opening 492A. The first adhesive portion can also define an opening (e.g., similar in size and shape to the opening 492A) such that the electrode 481 disposed on a bottom side of the portion 486 is accessible through the opening 492A. The first assembly 410 also includes a hydrogel portion 491.

The second assembly 420 includes a second upper housing 454, a portion 484 of the composite assembly 480, a second lower housing 494, and a second adhesive portion (not shown). The portion 484 can include any suitable electronic components (e.g., an energy storage device such as a coin cell battery). The second lower housing 494 defines an opening 494A such that an electrode 483 disposed on a bottom side of the portion 484 is accessible through the opening 494A. The second adhesive portion can also define an opening (e.g., similar in size and shape to the opening 494A) such that the electrode 483 disposed on a bottom side of the portion 484 is accessible through the opening 494A. The second assembly 420 also includes a hydrogel portion 493.

In some implementations, the composite assembly 480 includes a tab contact 488. The tab contact 488 can be integrally formed with the composite board of the composite assembly 480 and can be folded to contact the top of the energy storage device of the portion 484 as shown in FIG. 4. In some implementations, the energy storage device can be coupled to the composite board of the composite assembly 480 via a conductive adhesive. In some implementations, contacts of the energy storage device can be coupled to the composite board via spot welding.

The connecting member 430 includes a third upper housing 456, a portion 482 of the composite assembly 480, a third lower housing 496, and a third adhesive portion (not shown). The third lower housing 496 has a skin-facing surface 485 along the length of the portion 482. The portion 482 can include a composite board including an insulator and at least one conductive trace (e.g., a flexible printed circuit board). The insulator can include, for example, polyimide. The at least one conductive trace can include, for example, copper. In some implementations, the composite board can include a polyimide with double-sided copper conductors. In some implementations, the portion 482 can include multiple layers (e.g., two, three, or more layers) with each layer including at least one conductive trace. In some implementations, the portion 482 can include multiple layers including at least one conductive trace with each layer including at least one conductive trace coupled to another layer including at least one conductive trace via an insulative layer. In some implementations, the patch assembly 402 includes three conductive traces extending from the first assembly 410 to the second assembly 420. For example, a first conductive trace can extend from a positive side of the energy storage device of the portion 484 to the portion 486, a second conductive trace can extend from a negative side of the energy storage device of the portion 484 to the portion 486, and the third conductive trace can extend from the electrode 483 to the portion 486. Similarly as described above with reference to the connecting member 130, in some implementations the connecting member 430 (and/or the portion 482) may have a thickness equal to or less than 100 μm. In some implementations, the height of the connecting member 430 (and/or the portion 482) can be, for example, equal to or less than 36 μm. In some implementations, the spring constant of the connecting member 430 (and/or the portion 482) (in the X-direction) can increase proportionally to a cube of the thickness of the connecting member 430 (and/or the portion 482) and linearly with respect to the height of the connecting member 430 (and/or the portion 482). In some implementations, the third adhesive portion can cover the entire skin-facing surface 485 of the third lower housing 496.

As shown in FIG. 4, the first upper housing 452, the second upper housing 454, and the third upper housing 456 can collectively form a cover layer 450. The first lower housing 492, the second lower housing 494, and the third lower housing 496 can collectively form a bottom layer 490. The bottom layer 490 can be coupleable to a surface of a skin via the first adhesive portion, the second adhesive portion, and/or the third adhesive portion such that the bottom layer 490 secures the composite assembly 480 to the surface of the skin. In some implementations, the cover layer 450 (including the first upper housing 452, the second upper housing 454, and the third upper housing 456) can be monolithically or integrally formed. In some implementations, the bottom layer 490 (including the first lower housing 492, the second lower housing 494, and the third lower housing 496) can be monolithically or integrally formed. In some embodiments, the first adhesive portion, the second adhesive portion, and the third adhesive portion can be included in a continuous adhesive layer. The continuous adhesive layer can be shaped and sized similarly to the bottom layer 490 and disposed on the bottom surface of the bottom layer 490.

A frame assembly, such as any of the frame assemblies described herein, can be coupled to the patch assembly 402 via connectors coupled to the first assembly 410, the second assembly 420, and/or the connecting member 430 such that the frame assembly can maintain the patch assembly 402 in a first patch configuration. For example, the frame assembly can maintain an intended distance between the first assembly 410 and the second assembly 420 and a particular shape of the connecting member 430 such that, when the patch assembly 402 is coupled to a user's skin via adhesive, the first assembly 410 and the second assembly 420 are separated by the intended distance and the connecting member 430 conformally couples to the skin of the user. For instance, the first assembly 410 and the second assembly 420 may each include an electrode, and the frame assembly may be configured to maintain an electrode to electrode distance of the patch assembly 402 such that the electrodes can be properly spaced apart when the patch assembly 402 is coupled to a user's skin. Additionally, in some embodiments, an opening defined by the frame assembly can be shaped to correspond to an outer profile of at least a portion of the patch assembly disposed within the opening. For example, the connecting member 430 may have a sinusoidal shape and the opening defined by the frame assembly can have a sinusoidal shape corresponding to an outer profile of at least a portion of the connecting member 430.

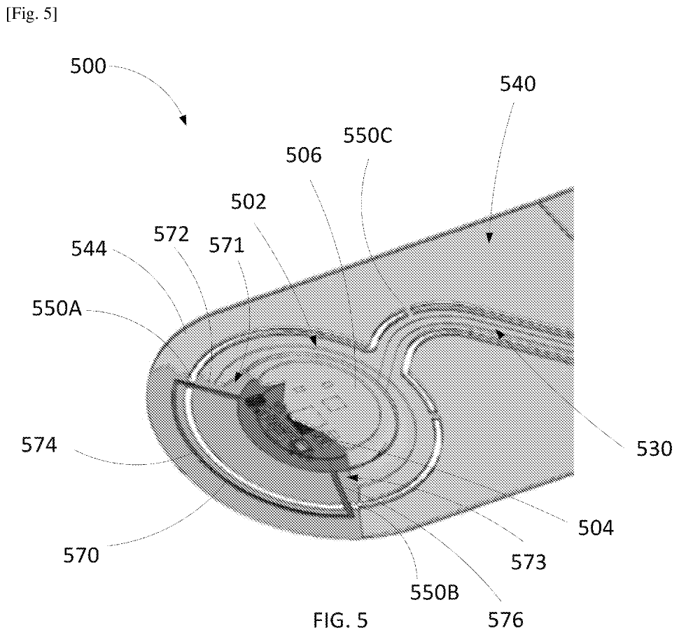

FIG. 5 is a perspective view of a portion of a system 500. Portions of the system 500 can be the same or similar in structure and/or function to any of the systems described herein. For example, the system 500 includes a frame assembly 540, a patch assembly 502, a conductor 570, and a set of connectors 550A-550C. The frame assembly 540 can be the same or similar in structure and/or function to any of the frame assemblies or frames described herein. The patch assembly 502 can include an electrical sub-assembly 504 that includes a composite assembly. The composite assembly can include, for example, a flexible PCB. Furthermore, the conductor 570 can include a portion of a composite assembly (e.g., a flexible PCB) that is coupled to the composite assembly of the electrical subassembly 504. For example, the conductor 570 can be formed by a copper trace etched in polyimide.

As shown in FIG. 5, the conductor 570 can form a loop including a first segment 572 extending across a first connector 550A, a second segment 574 disposed on the frame 540, and a third segment 576 extending across a second connector 550B. When the frame 540 is in the first frame configuration, the conductor 570 can form a closed circuit coupled to the electronics subassembly 504 such that energy and/or current can be sent through a first end 571 of the conductor component 570 and be received via a second end 573 of the conductor component 570. In some instances, this can cause a voltage at a portion of the electrical subassembly 504 to detect a voltage below a threshold. As shown in partial cut-away, the frame assembly 540 can include a top layer 544 and the patch assembly can include an upper housing 506. The top layer 544 and the upper housing 506 can cover the conductive component 570.

When the frame 540 transitions to the second frame configuration, the first segment 572 and/or the third segment 576 can be broken within or near the area of the first connector 550A and the second connector 550B. The remaining connectors from the set of connectors, such as the third connector 550C, can also be broken such that the frame 540 can be separated from the patch assembly 502. In some embodiments, the first segment 572 and the third segment 576 can be shaped with a particular width or thickness such that the first segment 572 and the third segment 576 break during separation of the frame assembly 540 from the patch assembly 502. For example, the first segment 572 and/or the third segment 576 can include a particular thickness or width in the area within or near the first connector 550A and/or the second connector 550B.

In some embodiments, the conductive component 570, or any of the conductive components described herein, can be formed via printing or coating a particle-type material. For example, the conductive component can be formed of a printed carbon or a printed silver loop. The printed carbon or printed silver can be cured to form a conducting granular network. The conducting granular network can be easier to break than a copper trace (e.g., of a flexible PCB) and can be printed with smaller thickness and width than a copper trace.

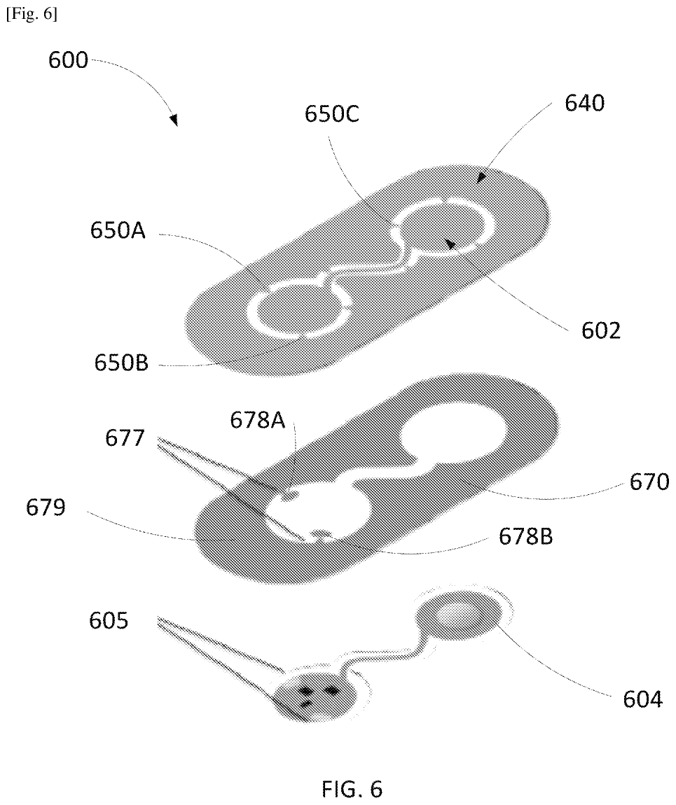

FIG. 6 is an exploded perspective view of a system 600. Portions of the system 600 can be the same or similar in structure and/or function to any of the systems described herein. For example, the system 600 includes a patch assembly 602 including an electronics subassembly 604, a frame assembly 640, and a set of connectors 650A-650C. The patch assembly 602 can be configured to be coupled to a patient via an adhesive portion (not shown).

The frame assembly 640 can have a first frame configuration in which the frame assembly 640 is coupled to the patch assembly 602 via the set of connectors 650A-650C and a second frame configuration in which each connector from the set of connectors 650A-650C is broken and the frame assembly 640 is separated from the patch assembly 602. The frame assembly 640 includes a conductive layer 670 having a conductive frame portion 679, a first coupling area 678A, and a second coupling area 678B. In some embodiments, the conductive layer 670 can be printed on a layer of the frame assembly 640, such as an underside of a top layer. The first coupling area 678A and the second coupling area 678B are coupled to the conductive frame portion 679 via a set of conductive connectors 677. Each conductive connector 677 from the set of conductive connectors can be included in a connector from the set of connectors 650A-650C. For example, a first conductive connector 677 can be included in a first connector 650A and a second conductive connector 677 can be included in a second connector 650B. Additional connectors from the set of connectors, such as a third connector 650C, can be free of conductive connectors 677.

The electronics subassembly 604 includes coupling areas 605 (e.g., a first coupling area and a second coupling area). The first coupling area 678A of the conductive layer 670 can be coupled to the first coupling area 605 of the electronics subassembly 604 and the second coupling area 678B of the conductive layer 670 can be coupled to the second coupling area 605 of the electronics subassembly 604. In some embodiments, the coupling areas 605 can include silver-plated contact pads. In some embodiments, the coupling areas 605 can be coupled to the first coupling area 678A and the second coupling area 678B via conductive adhesive. When the frame assembly 640 is in the first frame configuration, the conductive layer 670 can form a closed electrical circuit from the first coupling area 605 of the electronics subassembly 604 to the second coupling area 605 of the electronics subassembly 604 via the conductive layer 670.

Each conductive connector 677 from the set of conductive connectors can be configured to be broken when the frame assembly 670 is transitioned from the first frame configuration to the second frame configuration. Because, when in the second frame configuration, the conductive layer 670 is discontinuous from the first coupling area 678A to the second coupling area 678B, the electronics subassembly 604 can detect that the conductive layer 670 in combination with the electronics subassembly 604 does not form a closed circuit.

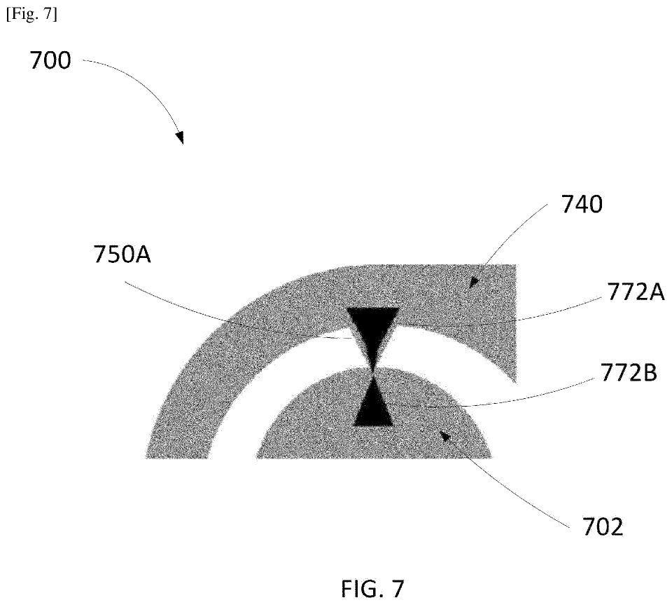

In some embodiments, a portion of the conductive component can have an hourglass shape having a first portion having a first width and a second portion having a second width. The second width can be less than the first width. For example, FIG. 7 is a schematic illustration of a portion of the system 700. Portions of the system 700 can be the same or similar in structure and/or function to any of the systems described herein. For example, the system 700 includes a patch assembly 702, a frame 740, and a connector 750A. The frame 740 has a first frame configuration in which the frame 740 is coupled to the patch assembly 702 via the connector 750A. The system 700 also includes a first conductive portion 772A and a second conductive portion 772B. The first conductive portion 772A and the second conductive portion 772B can be the same or similar in structure and/or function to any of the portion of conductive components extending across or near connectors described herein.

As shown in FIG. 7, the first conductive portion 772A and the second conductive portion 772B are tapered toward each other such that the first conductive portion 772A and the second conductive portion 772B collectively form an hourglass shape. The interface of the first conductive portion 772A and the second conductive portion 772B (e.g., the smallest portion of the hourglass shape) can be have a smaller width than other portions of the first conductive portion 772A or the second conductive portion 772B such that the interface represents the weakest portion of the first conductive portion 772A and the second conductive portion 772B. Thus, the first conductive portion 772A and the second conductive portion 772B are configured to separate at the interface of the first conductive portion 772A and the second conductive portion 772B when the connector 750A is broken from the patch assembly 702.

As shown in FIG. 7, connector 750A and the first conductive portion 772A can be configured to break from the patch assembly 702 and the second conductive portion 772B, respectively, at slightly different locations. For example, the interface of the first conductive portion 772A and the second conductive portion 772B can be disposed on the patch assembly 702 within an outer perimeter of the patch assembly 702. Thus, the risk of an electronic subassembly of the patch assembly 702 inadvertently being broken when the frame assembly 740 is transitioned to the second frame configuration is reduced. Additionally, a broken edge of the second conductive portion 772B can be disposed within an outer perimeter of the patch assembly 702 such that a user is unlikely to be harmed by the broken edge.

In some embodiments, the frame assembly is coupled to the patch assembly by the connection between the first conductive portion and the second conductive portion, but not via the connector. Similarly stated, a space or gap can be defined between the connector and the patch assembly (e.g., adjacent or proximal the interface of the first conductive portion and the second conductive portion) such that the frame assembly is not coupled to the patch assembly via the connector. No space or gap is defined between the first conductive portion and the second conductive portion such that the frame assembly is coupled to the patch assembly 702 via the first conductive portion and the second conductive portion. Thus, the connector 750A and the first conductive portion can be separated from the second conductive portion with reduced force compared to if the connector is coupled to the patch assembly and is required to be broken from the patch assembly (e.g., via tearing the connector from the patch assembly) during the separation of the first conductive portion from the second conductive portion.

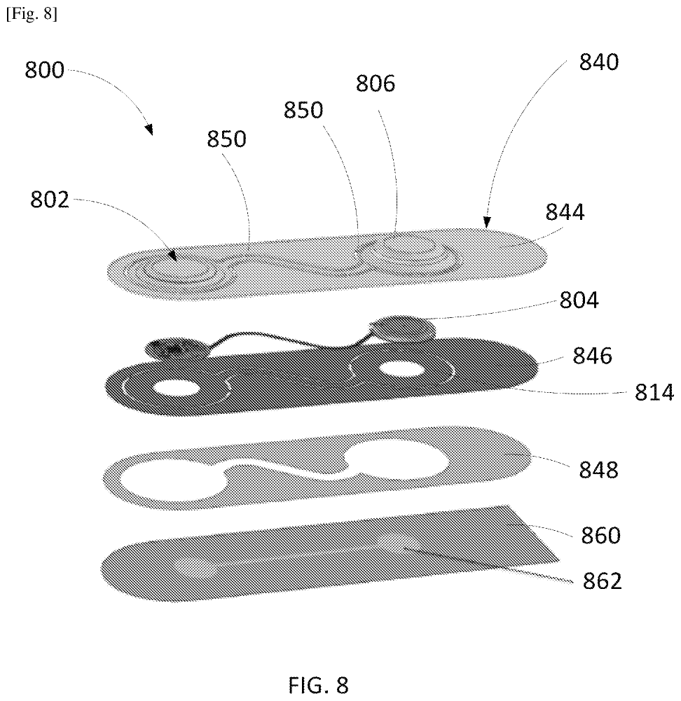

FIG. 8 is a perspective view of a system 800. Portions of the system 800 can be the same or similar in structure and/or function to any of the systems described herein. For example, the system 800 includes a frame assembly 840 and a patch assembly 802. The frame assembly 840 can be the same or similar in structure and/or function to any of the frames or frame assemblies described herein. For example, the frame assembly 840 includes a top layer 844, an adhesive layer 846, and a liner layer 848. The patch assembly 802 can be the same or similar in structure and/or function to any of the patch assemblies described herein. For example, the patch assembly 802 includes a housing 806, an electronics assembly 804, and an adhesive portion 814. In some embodiments, the adhesive portion 814 can form or include a lower housing portion of the patch assembly 802.

As shown in FIG. 8, the adhesive portion 814 can define openings such that electrodes of the patch assembly 802 are accessible via the openings and can contact a surface of a user when the patch assembly 802 is coupled to the user via the adhesive portion 814. In some embodiments, the electronics assembly 804 may be the same or similar in structure and/or function to the composite assembly 480 shown and described with respect to FIG. 4. The frame assembly 840 is configured to be coupled to the patch assembly 802 via a set of connectors 850. Each of the connectors 850 can include a housing portion and an adhesive portion such that the top layer 844, the housing 806, and the housing portion of each connector 850 can be formed as a single unitary layer, and the adhesive layer 846, the adhesive portion of the connectors 850, and the adhesive portion 814 can be formed as a single unitary layer. In some embodiments, the adhesive portion of each connector 850 can be omitted such that the connectors 850 break more easily.

The system 800 also includes a protective layer 860. The protective layer 860 includes a conductive component 862. The protective layer 860 is configured to be coupled to the patch assembly 802 via the adhesive portion 814 in a first protective layer configuration. The protective layer 860 is shaped and sized to contact and protect the bottommost surface of the frame assembly 840 prior to use of the system 800 (e.g., during storage). The protective layer 860 can be configured to protect at least the adhesive portion 814 and/or any hydrogel portion of the underside of the patch assembly 802. The protective layer 860 can be separated from the frame assembly 840 and the patch assembly 802 via, for example, peeling the protective layer 860 away from the adhesive portion 814 such that the protective layer 860 is in a second protective layer configuration. After the protective layer 860 is separated from the frame assembly 840 and the patch assembly 802, the frame assembly 840 and the patch assembly 802 can be coupled to a surface (e.g., a skin) of a user via the adhesive portion 814.

When the protective layer 860 is coupled to the patch assembly 840 in the first protective layer configuration, the conductive component 862 can be coupled to a first electrode of the electronics subassembly 804 and a second electrode of the electronics subassembly 804 such that the first electrode, the second electrode, and the conductive component 862 form a closed circuit. When the protective layer 860 is removed from the patch assembly 802 and transitioned to the second protective layer configuration, the first electrode and the second electrode no longer form a closed circuit with the conductive component 862. The first electrode and the second electrode can be configured to electrically contact a surface (e.g., skin) of a user when the patch assembly 802 is coupled to the surface. In response to the electronics assembly 804 detecting that the first electrode and the second electrode no longer form a closed circuit via the conductive component 862 (after removing the protective layer 860), the electronics assembly 804 can activate another component and/or operation of the electronics assembly 804. For example, measurements related to the surface (e.g., skin) of the patient can be taken by the electronics assembly 804 via the first electrode and the second electrode.

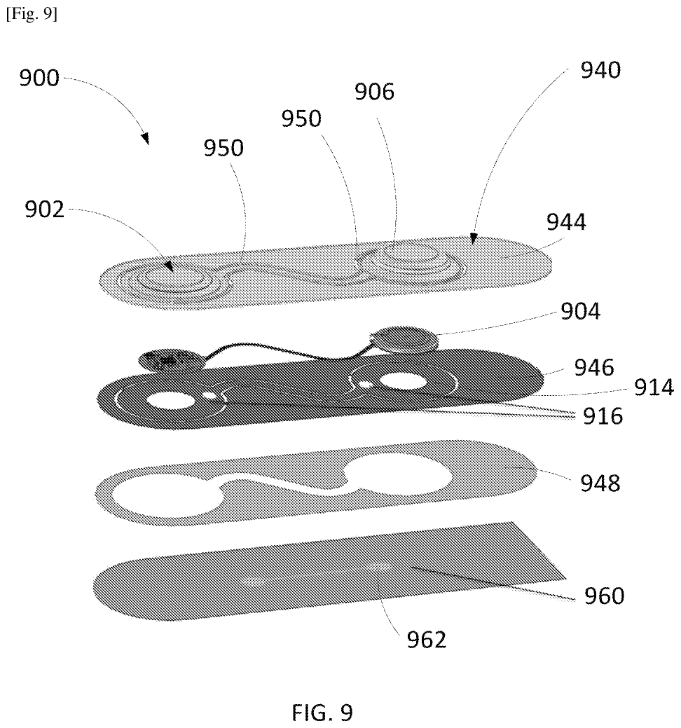

FIG. 9 is a perspective view of a system 900. Portions of the system 900 can be the same or similar in structure and/or function to any of the systems described herein, such as the system 800. For example, the system 900 includes a frame assembly 940 and a patch assembly 902. The frame assembly 940 can be the same or similar in structure and/or function to any of the frames or frame assemblies described herein. For example, the frame assembly 940 includes a top layer 944, an adhesive layer 946, and a liner layer 948. The patch assembly 902 can be the same or similar in structure and/or function to any of the patch assemblies described herein. For example, the patch assembly 902 includes a housing 906, an electronics assembly 904, and an adhesive portion 914. In some embodiments, the adhesive portion 914 can form or include a lower housing portion of the patch assembly 902. As shown in FIG. 9, the adhesive portion 914 can define openings such that electrodes of the patch assembly 902 are accessible via the openings and can contact a surface of a user when the patch assembly 902 is coupled to the user via the adhesive portion 914. In some embodiments, the electronics assembly 904 may be the same or similar in structure and/or function to the composite assembly 480 shown and described with respect to FIG. 4. The frame assembly 940 is configured to be coupled to the patch assembly 902 via a set of connectors 950. Each of the connectors 950 can include a housing portion and an adhesive portion such that the top layer 944, the housing 906, and the housing portion of each connector 950 can be formed as a single unitary layer, and the adhesive layer 946, the adhesive portion of the connectors 950, and the adhesive portion 914 can be formed as a single unitary layer. In some embodiments, the adhesive portion of each connector 950 can be omitted such that the connectors 950 break more easily.

The system 900 also includes a protective layer 960. The protective layer 960 includes a conductive component 962. The protective layer 960 is configured to be coupled to the patch assembly 902 via the adhesive portion 914 in a first protective layer configuration and to be removed from the patch assembly 902 in a second protective layer configuration. When the protective layer 960 is in the first protective layer configuration, the conductive component 962 is configured to be coupled to a first electrical connection and a second electrical connection of the electrical sub-assembly 904. For example, the adhesive portion 914 can define two openings 916 such that the first electrical connection and the second electrical connection are accessible by the conductive component 962 via the openings 916. Thus, with the protective layer 960 in the first protective layer configuration, the conductive component 962 can complete a closed electrical circuit in combination with the first electrical connection and the second electrical connection. The first electrical connection and the second electrical connection can be separate from a first electrode and a second electrode of the electronics subassembly 904, which are configured to be electrically coupled to a surface of a patient. When the protective layer 960 is removed from the patch assembly 902 and transitioned to the second protective layer configuration, the first electrical connection and the second electrical connection no longer form a circuit with the conductive component 962. In response to the electronics assembly 904 detecting that the first electrode and the second electrode no longer form a circuit, the electronics assembly 904 can activate another component and/or operation of the electronics assembly 904. For example, measurements related to the surface (e.g., skin) of the patient can be taken by the electronics assembly 904 via the first electrode and the second electrode.

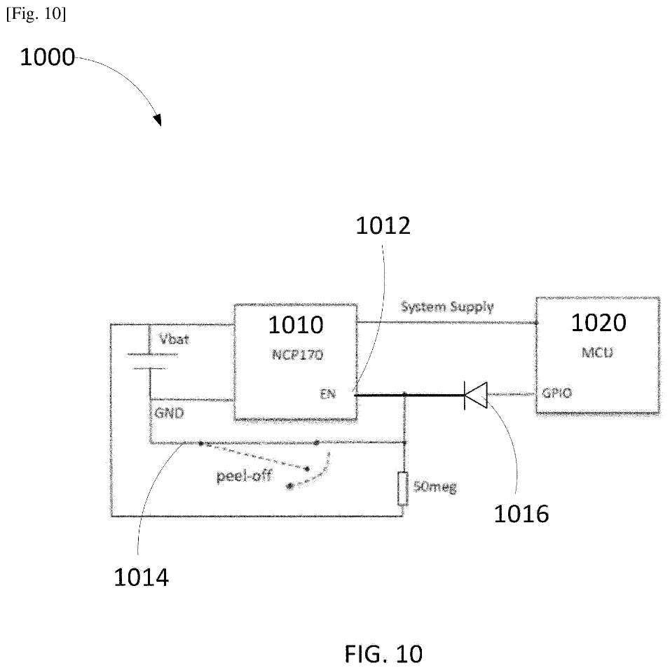

FIG. 10 is a schematic illustration of electrical components of a system 1000, according to an embodiment. Specifically, such electrical components can be implemented on a frame and/or patch assembly, similar to the electronics subassemblies and/or conductive components described herein. In some implementations, to implement the electronic detection of the conductor components breaking at sufficiently low power, as an example, a low dropout (LDO) regulator 1010 (e.g., a NCP170 regulator) can be used. The LDO regulator 1010 can provide a shutdown state which typically consumes around 100 nA. This is below an example target of 170 nA (corresponding to a CR1616 battery capacity 60 mAh drained 10% in four years). The LDO regulator 1010 can use a voltage of less than about 400 mV on an enable pin 1012 for deactivation, and a voltage of greater than about 1200 mV for activation. An activation conductor 1014 (e.g., similar to conductive components (e.g., 170, 170′ of FIGS. 1A and 1C, respectively, or 862, 962 of FIGS. 8 and 9, respectively) on a frame and/or protective layer) is used to pull the LDO regulator 1010 enable pin 1012 close to 0V before the patch assembly is released from the frame. A 50 Meg ohm resistor is used to pull the enable pin 1012 above 1200 mV after the activation conductor 1014 is broken (e.g., the patch assembly is released from the frame or protective layer). The activation conductor 1014 via the arrow designated as “peel-off” in FIG. 10.

In the deactivated state, the 50 Meg ohm resistor forms a voltage divider on the enable pin 1012, which allows a resistance of up to 7 Meg ohm along the activation conductor 1014, still assuring that less than 400 mV is present on the enable pin and the LDO regulator 1010 is deactivated. This allows a wide range of materials to be used to close the activation circuit, and reduces the need for precision high conductivity interconnect between the electronics activation terminals and the printed conductor. When the enable pin 1012 is forced low through the activation conductor 1014, the current through the 50 Meg ohm pull-up resistor will be about 60 nA resulting in a total current consumption under the example target of about 170 nA. In the activated state, the 50 Meg ohm resistor pulls the enable pin 1012 to about 3V, with a specified current of 10 nA into enable pin 1012.

If activation terminals of the electronics should find a parasitic conduction path, with a resistance of 40 Meg ohms, then the voltage that appears at the enable pin 1012 will fall below 1400 mV according to the voltage divider principle. This can lead to an unwanted deactivation of the patch after it is placed on the skin. Such parasitic reconnectivity can occur, for example, due to water (person in shower or swimming) or sweat (optionally combined with skin conductivity).

The permanent activation can be achieved reliably via the microcontroller (MCU) 1020. When the patch assembly is first activated, the MCU 1020 powers-up and performs processing to ensure that the activation is not a glitch. For example, the MCU 1020 can wait for confirmation of the patch assembly going into use, either using skin detection methods, or the attempt of wireless connection to another device. After the MCU 1020 determines the patch assembly is to be permanently activated, it then replaces the 50 Meg ohm pull-up resistor with one of much lower value, for example 10 kohm, provided through the MCU 1020 internal circuitry (not shown). This is achieved through a general purpose IO (GPIO) port 1022 as shown in FIG. 7. In the inactive and/or sleep state, the GIPO is protected from influencing the activation with a semiconductor barrier such as, for example, a diode 1016. The patch assembly can, for example, then operate to detect biological information via electrodes.

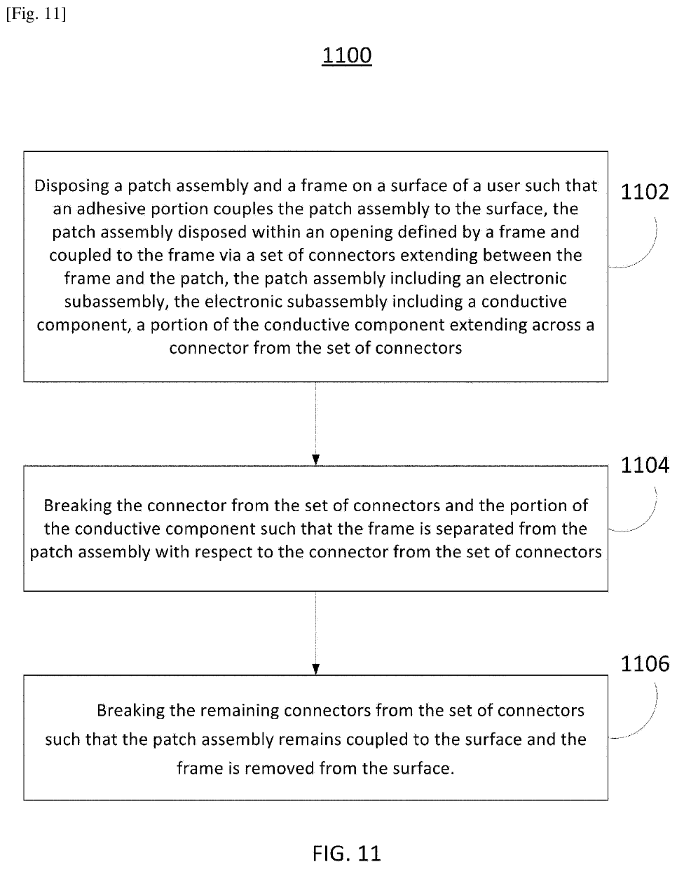

FIG. 11 is a flow chart representing a method 1100 of using certain of the systems described herein. The method 1100 includes, at 1102, disposing a patch assembly and a frame on a surface of a user such that an adhesive portion couples the patch assembly to the surface. The patch assembly can be disposed within an opening defined by the frame. The patch assembly can be coupled to the frame via a set of connectors extending between the frame and the patch. The patch assembly includes an electronic subassembly and includes a conductive component extending across a connector from the set of connectors.

At 1104, the connector from the set of connectors and the portion of the conductive component can be broken such that the frame is separated from the patch assembly with respect to the connector from the set of connectors. At 1106, each remaining connector from the set of connectors can be broken such that the patch assembly remains coupled to the surface and the frame is removed from the surface. In some embodiments, the electronic subassembly is configured to activate a sensor component of the electronic subassembly in response to the breaking of the portion of the conductive component.

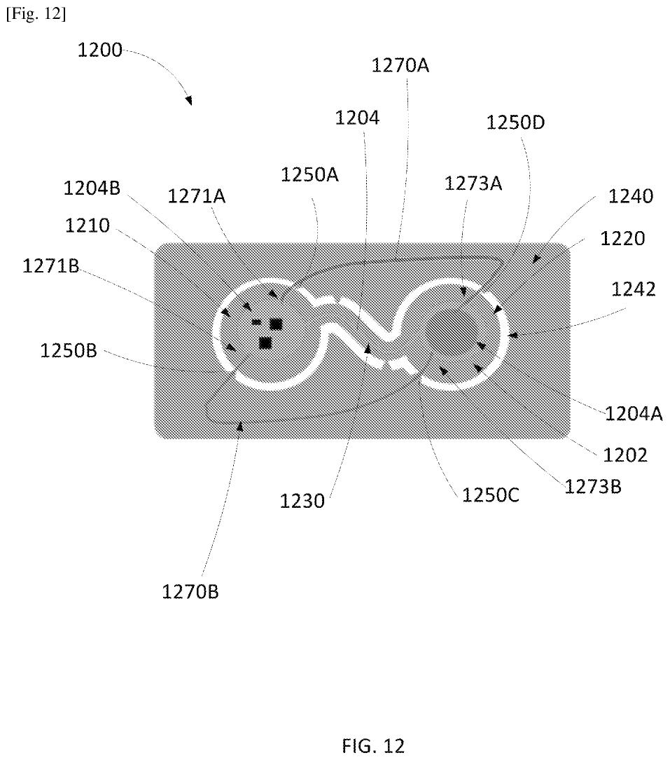

In some embodiments, a system can include a number of conductive components such that information can be gathered as to whether a frame assembly of the system has been entirely removed from the patch assembly. For example, FIG. 12 is a schematic illustration of a system 1200. Portions of the system 1200 can be the same or similar in structure and/or function to any of the systems described herein, such as the system 200, the system 100A, and/or the system 100B described above. For example, the system 1200 includes a patch assembly 1202, a frame 1240, a group of connectors including connectors 1250A, 1250B, 1250C and 1250D (also referred to as connectors 1250), a first conductive component 1270A and a second conductive component 1270B. The patch assembly 1202 can include a first assembly 1210, a second assembly 1220, and a connector assembly 1230. The patch assembly 1202 can be disposed within an opening 1242 defined by the frame 1240 such that a gap is defined between the patch assembly 1202 and the frame 1240. The patch assembly 1202, the frame 1240, the group of connectors 1250, and the conductive component 1270 can be the same or similar in structure and/or function to the patch assembly 102 or 102′, the frame 140 or 140′, the group of connectors 150 or 150′, and the conductive component 170 or 170′, respectively, described above with reference to the systems 100A and 100B of FIGS. 1A-1B and 1C-1D, respectively. The frame assembly 1240 can have a first frame configuration in which the frame assembly 1240 is coupled to the patch assembly 1202 via the group of connectors 1250 and a second frame configuration in which the group of connectors 1250 are broken and the frame assembly 1240 is separated from the patch assembly 1202. The patch assembly 1202 can include an electronics subassembly 1204 that includes a composite assembly. The composite assembly can include, for example, a flexible PCB. The first conductive component 1270A and the second conductive component 1270B can be electrically coupled to the electronics subassembly 1204. The electronics subassembly 1204 can be the same or similar in structure and/or function to any of the electronics subassemblies or electronics assemblies described herein.

In some implementations, the patch assembly 1202 can have a first patch configuration and a second patch configuration. For example, the patch assembly 1202 or a portion of the patch assembly 1202 may have a different shape (e.g., outer profile) and/or a different length in the first patch configuration compared to the second patch configuration. When the frame assembly 1240 is in the first frame configuration relative to the patch assembly 1202, the frame assembly 1240 can maintain the patch assembly 1202 in the first patch configuration via the group of connectors 1250. The frame assembly 1240 can be substantially inelastic. For example, the frame assembly 1240 can be substantially inelastic along its longitudinal axis such that, when in the first frame configuration when the frame assembly 1240 is coupled to the patch assembly 1202, the frame assembly 1240 (via the group of connectors 1250) can prevent the patch assembly 1202 from changing shape. For example, the frame assembly 1240 can prevent the patch assembly 1202 from transitioning between the first patch configuration and the second patch configuration.

The patch assembly 1202 can include a housing (not shown in FIG. 12) and an adhesive portion (not shown in FIG. 12). In some embodiments, the housing can include an upper housing portion (not shown in FIG. 12). In some embodiments, the housing can include an upper housing portion and/or a lower housing portion (not shown in FIG. 12). The frame assembly 1240 includes a top layer (not shown in FIG. 12). The top layer is coupled to the patch assembly 1202 in a first frame configuration via the connectors 1250. For example, the top layer can be coupled to the housing via the connectors 1250. In some embodiments, the top layer, the connectors 1250, and the housing can be formed of the same material. In some embodiments, the top layer, the connectors 1250, and the housing can be monolithically or integrally formed (e.g., formed of one sheet of material). In a second frame configuration, the frame assembly 1240 can be separated from the patch assembly 1202 via breaking the connectors 1250.

The first conductive component 1270A has a first end 1271A and a second end 1273A. The second conductive component 1270B has a first end 1271B and a second end 1273B. The first end 1271A and the second end 1273A of the first conductive component 1270A and the first end 1271B and the second end 1273B of the second conductive component 1270B are coupled to the electronics subassembly 1204. For example, the first end 1271A can be coupled to the electronics subassembly 1204 at a first location on the first assembly 1210 and the second end 1271B can be coupled to the electronics subassembly 1204 at a first location on the second assembly 1220, with a portion of the first conductive component 1270A disposed on the frame assembly 1240. The first end 1271B can be coupled to the electronics subassembly 1204 at a second location on the first assembly 1210 and the second end 1273B can be coupled to the electronics subassembly 1204 at a second location on the second assembly 1220, with a portion of the second conductive component 1270B disposed on the frame assembly 1240.

The first conductive component 1270A and the second conductive component 1270B can each form a continuous loop and/or a closed electrical circuit when the frame 1240 is in the first frame configuration. When the frame 1240 is transitioned from the first frame configuration to the second frame configuration, the first conductive component 1270A can be configured to break at the location of or near a first connector 1250A and a fourth connector 1250D to which the first conductive component 1270A is coupled, and the second conductive component 1270B can be configured to break at the location of or near a second connector 1250B and a third connector 1250C to which the second conductive component 1270B is coupled. When the frame 1240 is in the second frame configuration, at least a portion of the first conductive component 1270A and a portion of the second conductive component 1270B are broken such that the first conductive component 1270A and the second conductive component 1270B are discontinuous and neither forms a closed electrical circuit.

The electronics subassembly 1204 can be configured to detect whether the first conductive component 1270A and the second conductive component 1270B are continuous and/or discontinuous (e.g., fragmented due to being broken in an area proximate a connector from the set of connectors 1250). For example, when the frame 1240 is in the first frame configuration, a portion of the electronics subassembly 1204 can detect a voltage below a threshold associated with the first conductive component 1270A and a voltage below a threshold associated with the second conductive component 1270B. The electronics subassembly 1204 can remain in a sleep and/or inactive state when a voltage below a threshold associated with one or both of the first conductive component 1270A and the second conductive component 1270B is detected. For another example, the electronics subassembly 1204 can transmit electrical energy and/or current from a first portion 1204A of the electronics sub-assembly 1204 (e.g., from an energy storage device), to a second portion 1204B of the electronics subassembly 1204 via the first end 1271A of the first conductive component 1270A, the first conductive component 1270A, and the second end 1273A of the first conductive component 1270A such that the energy and/or current is transmitted through a closed circuit. The electronics subassembly 1204 can also transmit electrical energy and/or current from the first portion 1204A of the electronics subassembly 1204 (e.g., from an energy storage device) to a second portion 1204B of the electronics subassembly via the first end 1271B of the second conductive component 1270B, the second conductive component 1270B, and the second end 1273B of the second conductive component 1270A such that the energy and/or current is transmitted through a closed circuit. The electronics subassembly 1204 can be configured to detect that electrical energy and/or current is flowing through the first conductive component 1270A and/or the second conductive component 1270B such that the first conductive component 1270A and/or the second conductive component 1270B forms a continuous loop. For example, when the frame assembly 1240 is in the first frame configuration, a low voltage can be constantly, periodically and/or sporadically applied to the first conductive component 1270A and the second conductive component 1270B to identify that the patch assembly 1202 is connected to the frame assembly 1240 via each of the first connector 1250A, the second connector 1250B, the third connector 1250C, and the fourth connector 1250D. This can keep the remaining portions of the electronics subassembly 1204 on the patch assembly 1202 in a sleep and/or inactive state.

When the frame 1240 is in the second frame configuration, the connectors from the set of connectors 1250 and portions of the first conductive component 1270A and the second conductive component 1270B coupled to the connectors 150 are broken. Thus, energy and/or current is unable to be transmitted by the electronics subassembly 1204 through the first end 1271A to the second end 1273A of the first conductive component 1270A due to the first conductive component 1270A being discontinuous in the portions broken during separation of the frame assembly 1240 from the patch assembly 1202 (e.g., the first conductive component 1270A forms an open circuit when the frame assembly 1240 is in the second frame configuration). Similarly, energy and/or current is unable to be transmitted by the electronics subassembly 1204 through the first end 1271B to the second end 1273B of the second conductive component 1270B due to the second conductive component 1270B being discontinuous in the portions broken during separation of the frame assembly 1240 from the patch assembly 1202 (e.g., the second conductive component 1270B forms an open circuit when the frame assembly 1240 is in the second frame configuration). In some instances, when the first conductive component 1270A and/or the second conductive component 1270B is broken, the voltage at the portion of the electronics subassembly 1204 can increase, activating the electronics subassembly 1204. In some instances, the electronics sub-assembly 1204 can be configured to detect that the first conductive component 1270A or the second conductive component 1270B is discontinuous due to the energy and/or current flowing through the first conductive component 1270A and/or the second conductive component 1270B (from the first end 1271A or the first end 1271B, respectively) not being received by the electronics subassembly 1204 (via the second end 1273A or the second end 1273B). For example, in response to the electronics sub-assembly 1204 identifying that the first conductive component 1270A and the second conductive component 1270B are both broken and no longer form a closed electrical circuit, the remaining portions of the electronics subassembly 1204 can transition to an active state (e.g., to begin monitoring and/or sensing biological parameters of the user via electrodes).

In response to determining that the first conductive component 1270A and the second conductive component 1270B are discontinuous, in some embodiments, the electronics subassembly 1204 can be configured to actuate a component and/or operation of the electronics subassembly 1204. In some implementations, the energy and/or current provided to actuate a component and/or operation of the electronics subassembly 1204 can be provided at a greater power level than the power level of the energy and/or current provided to the first conductive component 1270A and/or the second conductive component 1270B prior to the first conductive component 1270A and the second conductive component 1270B being broken. For example, in some embodiments in which the electronics subassembly 1204 includes a number of electrodes configured to be coupled to a surface (e.g., skin) of a user, the electronics subassembly 1204 can actuate a component and/or operation of the electronics subassembly 1204 to measure electrical potential differences between locations on the surface to which the electrodes are coupled. In some embodiments, the electronics subassembly 1204 can include a sensor that can be actuated in response to the first conductive component 1270A and the second conductive component 1270B being broken. In some embodiments, the electronics subassembly 104 can be actuated to detect signals (e.g., electrocardiogram (EKG) signals, electroencephalogram (EEG) signals, electromyography (EMG) signals), to detect digital medicine within a user (e.g., wearer) of the patch assembly 1202, to detect signals from an ingested, implanted, or inserted device within a user, and/or to transmit information to an external communication device (e.g., a smart phone) and/or a remote server via any suitable communication method (e.g., Bluetooth(trademark), NFC, or WiFi(trademark)).

In some embodiments, to determine if the frame assembly 1240 has been only partially separated from the patch assembly 1202, the electronics subassembly 1204 can be configured to detect if only one of the first conductive component 1270A or the second conductive component 1270B has been transitioned from a continuous to a discontinuous configuration. For example, if only the first connector 1250A and/or the fourth connector 1250D have been broken but the second connector 1250B and the third connector 1250C remain unbroken, the electronics subassembly 1204 can determine that the second conductive component 1270B is still continuous and that one or both of the second connector 1250B and the third connector 1250C are unbroken through, for example, the same processes as described above. If the electronics subassembly 1204 determines that the frame assembly 1240 has been partially separated from the patch assembly 1202, the electronics subassembly 1204 can transmit information regarding the status of the separation and/or instructions for completing the separation of the frame assembly 1240 from the patch assembly 1202 to an external communication device for review by a user.

In some embodiments, the electronics subassembly 1204 may remain in a sleep and/or inactive state and not transition to an active or operational state unless both of the first conductive component 1270A and the second conductive component 1270B have been transitioned from a continuous to a discontinuous configuration (e.g., through breaking all of the connectors 1250). Thus, the electronics subassembly 1204 will remain in a sleep and/or inactive state if a connector of the connectors 1250 is inadvertently broken and/or if the frame assembly 1240 has not been properly removed from the patch assembly 1202.

Although the system 1200 is shown and described as including two conductive components, in some embodiments, the system can include any suitable number of conductive components. For example, the system can include three or more conductive components and/or a conductive component associated with each connector 1250 such that the electronics subassembly 1204 can provide data (e.g., to an external communication device) as to the continuity or discontinuity status of each connector 1250. For example, a conductive component can be disposed with two portions on each connector 1250 similarly as shown with respect to conductive component 170 and connector 150A in FIG. 1A such that the status of each connector 1250 can be communicated to an external communication device by the electronics subassembly 1204 upon each connector 1250 being broken.

In some embodiments, rather than the electronics subassembly 1204 being activated by the connectors 1250 being broken, the electronics subassembly 1204 can be activated by an external communication device (not shown). For example, the electronics subassembly 1204 can be configured to include wireless connectivity such as Bluetooth(trademark) or NFC. The external communication device can activate the electronics subassembly 1204 (e.g., via a Bluetooth(trademark) or NFC connection). For example, when the external communication device is brought within range of the electronics subassembly (e.g., a few centimeters), the external communication device can wirelessly provide power to the electronics subassembly 1204 (e.g., via an NFC antenna (not shown in FIG. 12) on the electronics subassembly 1204, the patch assembly 1202 and/or the frame assembly 1240). Such power can activate the electronics subassembly 1204 (e.g., transition the electronics subassembly 1204 from a sleep state to an active state). After activation, the electronics subassembly 1204 can provide status updates to the external communication device (e.g., via the Bluetooth(trademark) or NFC connection). For example, the electronics subassembly 1204 can provide information as to whether and/or how many of the connectors 1250 have been broken such that the external communication device can provide instructions for completing the separation of the frame assembly 1240 from the patch assembly 1202 to a user (e.g., to the external communication device via the Bluetooth(trademark) or NFC connection) if needed.

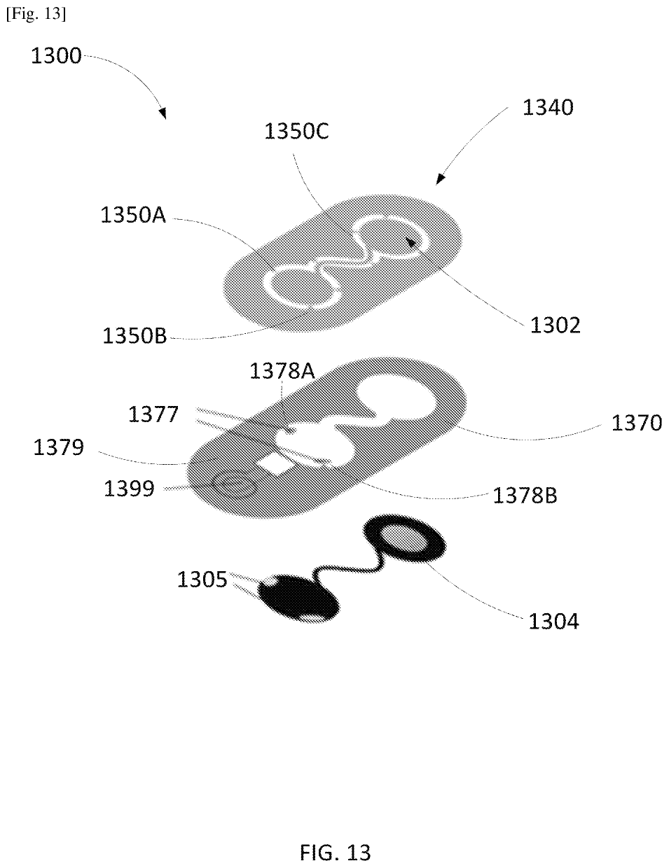

In some embodiments, a frame assembly of a system can include an NFC antenna such that a patch assembly of the system can be activated by an external communication device via the NFC antenna and/or paired with the external communication device via the NFC antenna prior to separation of the frame assembly from the patch assembly without the patch assembly needing to be sufficiently large to house the NFC antenna. For example, FIG. 13 is an exploded perspective view of a system 1300. Portions of the system 1300 can be the same or similar in structure and/or function to any of the systems described herein. For example, the system 1300 includes a patch assembly 1302 including an electronics subassembly 1304, a frame assembly 1340, and a set of connectors including connectors 1350A-1350C (collectively referred to herein as the set of connectors 1350 or the connectors 1350). The patch assembly 1302 can be configured to be coupled to a patient via an adhesive portion (not shown). The electronics subassembly 1304 can be the same or similar in structure and/or function to any of the electronics subassemblies or electronics assemblies described herein.

The frame assembly 1340 can have a first frame configuration in which the frame assembly 1340 is coupled to the patch assembly 1302 via the set of connectors 1350 and a second frame configuration in which each connector from the set of connectors 1350 is broken and the frame assembly 1340 is separated from the patch assembly 1302. The frame assembly 1340 includes a conductive layer 1370 having a conductive frame portion 1379, a first coupling area 1378A, and a second coupling area 1378B. In some embodiments, the conductive layer 1370 can be printed on a layer of the frame assembly 1340, such as an underside of a top layer. The first coupling area 1378A and the second coupling area 1378B are coupled to the conductive frame portion 1379 via a set of conductive connectors 1377. Each conductive connector 1377 from the set of conductive connectors can be included in a connector from the set of connectors 1350. For example, a first conductive connector 1377 can be included in a first connector 1350A and a second conductive connector 1377 can be included in a second connector 1350B. Additional connectors from the set of connectors 1350, such as a third connector 1350C, can be free of conductive connectors 1377.

The electronics subassembly 1304 includes coupling areas 1305 (e.g., a first coupling area and a second coupling area). The first coupling area 1378A of the conductive layer 1370 can be coupled to the first coupling area 1305 of the electronics sub-assembly 1304 and the second coupling area 1378B of the conductive layer 1370 can be coupled to the second coupling area 1305 of the electronics subassembly 1304. In some embodiments, the coupling areas 1305 can include silver-plated contact pads. In some embodiments, the coupling areas 1305 can be coupled to the first coupling area 1378A and the second coupling area 1378B via conductive adhesive. When the frame assembly 1340 is in the first frame configuration, the conductive layer 1370 can form a closed electrical circuit from the first coupling area 1305 of the electronics sub-assembly 1304 to the second coupling area 1305 of the electronics subassembly 1304 via the conductive layer 1370.

As shown in FIG. 13, the frame assembly 1340 can include an NFC receiver 1399. For example, the NFC receiver 1399 can be disposed on or coupled to the conductive layer 1370 and configured to be coupled to the electronics subassembly 1304 via the conductive frame portion 1379 and the first and second coupling areas 1305 of the electronics subassembly 1304. When the frame assembly 1340 is in the first frame configuration and the electronics subassembly 1304 has not been activated, the electronics subassembly 1304 can be in an inactive, low power or sleep state. To activate the electronics subassembly 1304, an external communication device (e.g., a smart phone) with NFC communication capabilities can be disposed within close proximity with the system 1300 (e.g., within about 4 cm). Activation power can be provided by the external communication device to the electronics subassembly 1304 via the NFC receiver 1399. The NFC receiver 1399 can be configured to detect the presence of the external communication device and can initiate activation of the electronics sub-assembly 1304 in response to receiving the activation power from the external communication device. In some embodiments, the external communication device can be configured to transmit an activation code (e.g., a unique activation code or number) to the NFC receiver 1399 prior to the NFC receiver 1399 initiating activation of the electronics subassembly 1304. The NFC receiver 1399 and/or another component of the electronics subassembly 1304 (e.g., a processor) can determine if the activation code matches an expected activation code, and, if so, can initiate activation of the electronics subassembly 1304. If the activation code does not match an expected activation code, then the NFC receiver 1399 and/or another component of the electronics subassembly 1304 can take no action. In response to being activated, the electronics subassembly 1304 can initiate activation of another component (e.g., a sensor component) and/or operation of the electronics subassembly 1304. For example, measurements related to the surface (e.g., skin) of the patient can be taken by the electronics subassembly 1304 via a first electrode and a second electrode of the patch assembly 1302. The electronics subassembly 1304 can draw more power from a power supply of the electronics subassembly 1304 after activation than when the electronics subassembly 1304 is in the inactive, low power or sleep state. Activating the electronics subassembly 1304 via NFC using an external communication device allows the electronics subassembly 1304 to remain in a low power state prior to activation, since no on-board power is needed for activation (as power can be wirelessly provided via the NFC connection). Thus, the system 1300 can have a long shelf life due to the low battery drain during storage.

In some embodiments, the external communication device and the electronics sub-assembly 1304 can exchange identification information via the NFC receiver 1399 to facilitate pairing (e.g., Bluetooth(trademark) pairing using Bluetooth Low Energy (BLE)). In some embodiments, the external communication device can automatically (e.g., without user action) pair with the system 1300 via the NFC receiver 1399 (e.g., after activation of the electronics subassembly 1304 by the external communication device via the NFC receiver 1399). Similarly stated, the NFC connection established between the NFC receiver 1399 and the external communication device can be used to automatically establish Bluetooth(trademark) communication and Bluetooth(trademark) pairing between the external communication device and the electronics subassembly 1304. Automatic pairing using the NFC connection reduces the risk of pairing the external communication device to another activated patch.

After the external communication device has been used to activate the electronics subassembly 1304 of the patch 1302 via the NFC receiver 1399 and/or the external communication device has been paired with the electronics subassembly 1304, the frame assembly 1340, including the NFC receiver 1399, can be removed from the patch assembly 1302. Thus, although the electronics subassembly 1304 of the patch assembly 1302 can be NFC-activated and BLE-paired, the NFC receiver 1399 does not need to remain coupled to the patch assembly 1302 for continued use of the patch assembly 1302. The patch assembly 1302 can continue to communicate with the external communication device and/or a remote server (e.g., via the external communication device) using BLE communication via BLE communication components included in the electronics subassembly 1304 of the patch assembly 1302.

Each conductive connector 1377 from the set of conductive connectors can be configured to be broken when the frame assembly 1370 is transitioned from the first frame configuration to the second frame configuration. In some embodiments, the electronics subassembly 1304 can detect that the conductive layer 1370 in combination with the electronics subassembly 1304 does not form a closed circuit in the second frame configuration due to the conductive layer 1370 being discontinuous from the first coupling area 1378A to the second coupling area 1378B. The electronics sub-assembly 1304 can transmit information to the external communication device and/or a remote server indicating whether the frame assembly 1370 has been separated from the patch assembly 1302 based on the detection of whether the conductive layer 1370 forms a closed circuit or an open circuit. This information can be used by the external communication device to determine whether the patch assembly 1302 has been applied and whether the frame assembly 1340 has been removed.

In some embodiments, the external communication device can be paired with and/or used to activate the electronics subassembly 1304 before and/or after the patch assembly 1302 has been coupled to the skin of a user. In some embodiments, upon the external communication device pairing with the electronics subassembly 1304, the external communication device can provide instructions regarding how to apply the patch assembly 1302 to a user's skin (e.g., instructing how to remove a liner from the patch assembly 1302 and/or where or how to apply the patch assembly 1302 to a skin surface) and/or how to remove the patch assembly 1302 from the frame assembly 1340 (e.g., identifying a hinge portion of the frame assembly 1340 and/or a direction to pull the hinge portion relative to the patch assembly 1302). The external communication device can also indicate to the user (e.g., via a user interface or display of the external communication device) when the frame assembly 1340 has been properly removed from the patch assembly 1302 (e.g., when each of the connectors 1350 and/or conductive components (described below) have been broken).

In some embodiments, the system 1300 can include a number of conductive components (e.g., conductive components 1270A and 1270B) to detect whether the frame assembly 1340 is in the first frame configuration or the second configuration, similarly as described above with respect to the system 1200. For example, rather than including the conductive layer 1370 being conductive across a surface of the conductive layer 1370, the conductive layer can include a number of conductive components extending across the connectors 1350. The NFC receiver 1399 can be coupled to the coupling areas 1378A and 1378B and the coupling areas 1305 via additional electrical components (e.g., conductive components).

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

In some embodiments, the systems (or any of its components) described herein can include a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Although various embodiments have been described as having particular features and/or combinations of components, other embodiments are possible having a combination of any features and/or components from any of the embodiments where appropriate.

In some embodiments, a system includes a patch assembly, a frame, and a conductive component. The patch assembly is configured to be coupled to a patient via an adhesive portion. The patch assembly includes an electronics subassembly. The frame has a first frame configuration in which the frame is coupled to the patch assembly via a plurality of connectors and a second frame configuration in which the plurality of connectors are broken and the frame is separated from the patch assembly. The conductive component has a first end and a second end. The first end and the second end is coupled to the electronics subassembly. The conductive component forms a continuous loop when the frame is in the first frame configuration. A portion of the conductive component is broken when the frame is in the second frame configuration such that the conductive component is discontinuous between the first end and the second end. The portion of the conductive component is at least partially disposed on a connector from the plurality of connectors when the frame is in the first frame configuration.

In some embodiments, the conductive component has a first segment, a second segment, and a third segment. The first segment is disposed on a first connector from the plurality of connectors, the second segment disposed on the frame, and the third segment disposed on a second connector from the plurality of connectors. The first segment is disposed to break into a first portion and a second portion when the first connector is broken.

In some embodiments, the electronics subassembly is configured to detect that the portion of the conductive component is broken and, in response to detecting that the portion of the conductive component is broken, to activate a sensor component of the electronics subassembly such that an operation of the electronics subassembly is initiated.

In some embodiments, the electronics subassembly provides energy through the conductive component at a first power level when the conductive component forms a continuous loop and provides energy to the sensor component at a second power level when the conductive component is discontinuous. The second power level is greater than the first power level.

In some embodiments, the electronics subassembly includes an energy storage device.

In some embodiments, the connector from the plurality connectors is a first connector, and a second connector from the plurality of connectors is not coupled to the conductive component.

In some embodiments, each connector from the plurality of connectors is tapered toward the patch assembly and the portion of the conductive component has an hourglass shape.

In some embodiments, the portion of the conductive component has an hourglass shape having a first portion having a first width and a second portion having a second width less than the width of the first portion.

In some embodiments, the electronics subassembly includes a first portion of a printed circuit board and the conductive component includes a second portion of the printed circuit board.

In some embodiments, a system includes a patch assembly and a frame assembly. The patch assembly is configured to be coupled to a patient via an adhesive portion. The patch assembly includes an electronics subassembly. The electronics subassembly includes a first coupling area and a second coupling area. The frame assembly has a first frame configuration in which the frame is coupled to the patch assembly via a plurality of connectors and a second frame configuration in which the plurality of connectors are broken and the frame is separated from the patch assembly. The frame assembly includes a conductive layer having a conductive frame portion, a first coupling area, and a second coupling area. The first coupling area and the second coupling area are coupled to the conductive frame portion via a set of conductive connectors. Each conductive connector from the set of conductive connectors are included in a connector from the plurality of connectors. The first coupling area of the conductive layer is coupled to the first coupling area of the electronics subassembly. The second coupling area of the conductive layer is coupled to the second coupling area of the electronics subassembly. The conductive layer forms an electrical circuit from the first coupling area of the electronics subassembly to the second coupling area of the electronics subassembly when the frame is in the first frame configuration. Each conductive connector from the set of conductive connectors is broken when the frame is in the second frame configuration such that the conductive layer is discontinuous between the first coupling area of the conductive layer and the second coupling area of the conductive layer.

In some embodiments, a system includes a patch assembly and a protective layer. The patch assembly is configured to be coupled to a user via an adhesive portion. The patch assembly includes an electronics subassembly. The protective layer includes a conductive component. The protective layer is coupled to the adhesive portion in a first protective layer configuration and removed from the patch assembly in a second protective layer configuration. The conductive component is coupled to the electronics subassembly such that energy can be conducted from a first component of the electronics subassembly to a second component of the electronics subassembly through the conductive component in the first protective layer configuration. The conductive component is not coupled to the electronics subassembly such that no energy is conducted from the first component of the electronics subassembly to a second component of the electronics subassembly in the second protective layer configuration.

In some embodiments, a system further comprises a frame having a first frame configuration in which the frame is coupled to the patch assembly via a plurality of connectors and a second frame configuration in which the plurality of connectors are broken and the frame is separated from the patch assembly.

In some embodiments, the protective layer is disposed in contact with a bottom surface of the frame.

In some embodiments, the first component of the electronics subassembly is a first electrode and the second component of the electronics subassembly is a second electrode. The first electrode and the second electrode are configured to couple to a surface of a user when the patch assembly is coupled to the surface.

In some embodiments, the electronic subassembly includes a first electrode and a second electrode. The first electrode and the second electrode are configured to couple to a surface of a user when the patch assembly is coupled to the surface.

In some embodiments, a method includes disposing a patch assembly and a frame on a surface of a user such that an adhesive portion couples the patch assembly to the surface. The patch assembly is disposed within an opening defined by a frame and coupled to the frame via a plurality of connectors extending between the frame and the patch. The patch assembly includes an electronic subassembly including a conductive component. A portion of the conductive component extends across a connector from the plurality of connectors. The connector from the plurality of connectors and the portion of the conductive component can be broken such that the frame is separated from the patch assembly with respect to the connector from the plurality of connectors. The remaining connectors from the plurality of connectors can be broken such that the patch assembly remains coupled to the surface and the frame is removed from the surface.

In some embodiments, the electronic subassembly is configured to activate a sensor component of the electronic subassembly in response to the breaking of the portion of the conductive component.

In some embodiments, a portion of a protective layer can be separated from a bottom surface of the patch assembly such that the protective layer is decoupled from an adhesive portion of the patch assembly.

1. A system, comprising:
a patch assembly configured to be coupled to a patient via an adhesive portion, the patch assembly including an electronics subassembly;
a frame having a first frame configuration in which the frame is coupled to the patch assembly via a plurality of connectors and a second frame configuration in which the plurality of connectors are broken and the frame is separated from the patch assembly; and
a conductive component having a first end and a second end, the first end and the second end coupled to the electronics subassembly, the conductive component forming a continuous loop when the frame is in the first frame configuration, a portion of the conductive component being broken when the frame is in the second frame configuration such that the conductive component is discontinuous between the first end and the second end, the portion at least partially disposed on a connector from the plurality of connectors when the frame is in the first frame configuration.

2. The system of claim 1, wherein the conductive component has a first segment, a second segment, and a third segment, the first segment disposed on a first connector from the plurality of connectors, the second segment disposed on the frame, and the third segment disposed on a second connector from the plurality of connectors, the first segment disposed to break into a first portion and a second portion when the first connector is broken.

3. The system of claim 1, wherein the electronics subassembly is configured to detect that the portion of the conductive component is broken and, in response to detecting that the portion of the conductive component is broken, to activate a sensor component of the electronics subassembly such that an operation of the electronics subassembly is initiated.

4. The system of claim 3, wherein the electronics subassembly provides energy through the conductive component at a first power level when the conductive component forms a continuous loop and provides energy to the sensor component at a second power level when the conductive component is discontinuous, the second power level being greater than the first power level. energy storage device.

6. The system of claim 1, wherein the connector from the plurality connectors is a first connector, further comprising a second connector from the plurality of connectors that is not coupled to the conductive component.

7. The system of claim 1, wherein the each connector from the plurality of connectors is tapered toward the patch assembly and the portion of the conductive component has an hourglass shape.

8. The system of claim 7, wherein the portion of the conductive component having an hourglass shape has a first portion having a first width and a second portion having a second width less than the width of the first portion.

9. The system of claim 1, wherein the electronics subassembly includes a first portion of a printed circuit board and the conductive component includes a second portion of the printed circuit board.

10. A system, comprising:
a patch assembly configured to be coupled to a patient via an adhesive portion, the patch assembly including an electronics subassembly, the electronics subassembly including a first coupling area and a second coupling area; and
a frame assembly having a first frame configuration in which the frame is coupled to the patch assembly via a plurality of connectors and a second frame configuration in which the plurality of connectors are broken and the frame is separated from the patch assembly, the frame assembly including a conductive layer having a conductive frame portion, a first coupling area, and a second coupling area, the first coupling area and the second coupling area coupled to the conductive frame portion via a set of conductive connectors, each conductive connector from the set of conductive connectors included in a connector from the plurality of connectors, the first coupling area of the conductive layer coupled to the first coupling area of the electronics subassembly, the second coupling area of the conductive layer coupled to the second coupling area of the electronics subassembly, the conductive layer forming an electrical circuit from the first coupling area of the electronics subassembly to the second coupling area of the electronics subassembly when the frame is in the first frame configuration, each conductive connector from the set of conductive connectors broken when the frame is in the second frame configuration such that the conductive layer is discontinuous between the first coupling area of the conductive layer and the second coupling area of the conductive layer.

11. A system, comprising:
a patch assembly configured to be coupled to a user via an adhesive portion, the patch assembly including an electronics subassembly; and
a protective layer including a conductive component, the protective layer coupled to the adhesive portion in a first protective layer configuration and removed from the patch assembly in a second protective layer configuration, the conductive component coupled to the electronics subassembly such that energy can be conducted from a first component of the electronics subassembly to a second component of the electronics subassembly through the conductive component in the first protective layer configuration, the conductive component not coupled to the electronics subassembly such that less energy is conducted from the first component of the electronics subassembly to a second component of the electronics subassembly in the second protective layer configuration.

12. The system of claim 11, further comprising:
a frame having a first frame configuration in which the frame is coupled to the patch assembly via a plurality of connectors and a second frame configuration in which the plurality of connectors are broken and the frame is separated from the patch assembly.

13. The system of claim 12, wherein the protective layer is disposed in contact with a bottom surface of the frame.

14. The system of claim 11, wherein the first component of the electronics subassembly is a first electrode and the second component of the electronics subassembly is a second electrode, the first electrode and the second electrode configured to couple to a surface of a user when the patch assembly is coupled to the surface.

15. The system of claim 11, wherein the electronic subassembly includes a first electrode and a second electrode, the first electrode and the second electrode configured to couple to a surface of a user when the patch assembly is coupled to the surface.

16. A method, comprising:
disposing a patch assembly and a frame on a surface of a user such that an adhesive portion couples the patch assembly to the surface, the patch assembly disposed within an opening defined by a frame and coupled to the frame via a plurality of connectors extending between the frame and the patch assembly, the patch assembly including an electronic sub-assembly, the electronic subassembly including a conductive component, a portion of the conductive component extending across a connector from the plurality of connectors;
breaking the connector from the plurality of connectors and the portion of the conductive component such that the frame is separated from the patch assembly with respect to the connector from the plurality of connectors; and
breaking the remaining connectors from the plurality of connectors such that the patch assembly remains coupled to the surface and the frame is removed from the surface.

17. The method of claim 16, wherein the electronic subassembly is configured to activate a sensor component of the electronic sub-assembly in response to the breaking of the portion of the conductive component.

18. The method of claim 16, further comprising separating a portion of a protective layer from a bottom surface of the patch assembly such that the protective layer is decoupled from an adhesive portion of the patch assembly.
